(19) United States
(12) Patent Application Publication
(10) Pub. No.: US 2008/0070237 A1
(43) Pub. Date: Mar. 20, 2008

(54) Npas3 Mutant Mice and Uses for Screening and Testing Therapies for Schizophrenia and Related Neurological Disorders

(76) Inventors: Eric W. Brunskill, Cincinnati, OH (US); Steven S. Potter, Cincinnati, OH (US)

Correspondence Address: HASSE & NESBITT LLC, 8837 CHAPEL SQUARE DRIVE, SUITE C, CINCINNATI, OH, 45249, US

(21) Appl. No.: 10/568,160

(22) PCT Filed: Aug. 11, 2004
(86) PCT No.: PCT/US04/26052

§ 371 Date: Jan. 15, 2007

Related U.S. Application Data
(60) Provisional application No. 60/494,197, filed on Aug. 11, 2003.
(60) Provisional application No. 60/481,952, filed on Jan. 26, 2004.

(51) Int. Cl. A01K 67/00; C12N 5/10; C12Q 1/68; G01N 33/00

(52) U.S. Cl. 435/6; 435/354; 800/3; 800/9

(57) ABSTRACT

A transgenic mouse with a gene-targeted mutation of the Npas3 gene, and a model of schizophrenia and related neurological disorders such as Obsessive-Compulsive Disorder, Tourette's Syndrome, and bipolar disorders, and other neurological disorders affecting locomotor activity, including Parkinson's Disease. A method is also provided for use of the Npas3-deficient mouse for testing the efficacy of a biologically active agent as a treatment for schizophrenia and related neurological disorders. The methods further includes uses of cells and cell lines derived from an Npas3-deficient mouse to screen biologically active agents that can alter biochemical pathways involved in schizophrenia and related neurological disorders, including those affecting locomotion.

### TECHNICAL FIELD

The present invention relates to a transgenic mouse model for schizophrenia and related neurological disorders, including those affecting locomotion. In particular, the invention relates to a classification of disorders known as schizophrenia and schizoaffective disorder. The invention further relates to methods for screening biologically active agents that can alter biochemical pathways involved in neurological diseases, and to test pharmacological therapies for schizophrenia and related neurological disorders, including those affecting locomotion.

### BACKGROUND

Schizophrenia is a devastating psychiatric illness that affects approximately 1% of the world population irrespective of ethnic, economic, or cultural boundaries. See Rowley et al. (2001) Jour. Medicinal Chem. 44 (4): 477-501. Only about 25% of patients recover to any significant extent within 5 years of starting treatment with currently available drug therapies. Approximately 65% of patients have recurring problems over many years. The remaining 10-15% of patients develops long-term incapacity and around 15% of these commit suicide. There are substantial costs, direct and indirect, incurred by this disorder including those of drug treatment, residential accommodation, physician and other healthcare services, and loss of productivity in the workplace. Clinical symptoms are apparent relatively early in life, generally occurring between the ages of 15 and 45. They are characterized by the presence of positive symptoms, for example auditory hallucinations, disorganized thoughts, delusions, and irrational fears, and negative symptoms, including social withdrawal, diminished affect, poverty of speech, lack of energy, and the inability to experience pleasure. In addition, schizophrenic patients may suffer cognitive deficits including impaired attention, verbal fluency, memory recall, and executive function. The diagnostic criteria for this disease have been established by the International Classification of Diseases, 10^{th} ed. Geneva: WHO, 1994; and the Diagnostic and Statistical Manual of Mental Disorders, 4th ed. Washington, D.C.: Am. Psych. Press, 1994. In addition to the classical diagnosis of schizophrenia, many subtypes of schizophrenia and schizoaffective disorders have also been proposed. These psychiatric illnesses also have overlapping symptoms with other related neurological disorders, particularly Tourette's Syndrome, bipolar disorders, obsessive compulsive disorders, and disorders involving locomotor activity, such as Parkinson's Disease.

A number of studies have suggested that the development of symptoms is the result of genetic factors and environmental perturbations that alter brain development and function. For decades, the primary defect associated with schizophrenia has been attributed to dysfunctional dopamine signaling, since many effective anti-psychotic therapies target these signaling pathways. Despite its success in describing some aspects of schizophrenia, this hypothesis has yielded to a more encompassing view in which alterations of complex neural circuits, involving multiple neurochemical signaling molecules such as glutamate, γ-amino butyric acid (GABA) and serotonin, are also thought to be involved in the development of schizophrenia.

During the past 10 years, the limited efficacy of drugs designed to be specific for high-affinity binding to preselected dopamine receptors (typical antipsychotics) has shifted the attention of clinical investigators to drugs with a broader spectrum of receptor action, which, in addition to dopamine receptors, also have a high affinity for serotonin, noradrenalin, acetylcholine, glutamate and GABA receptors. See Costa et al. (2002) Current Opinion in Pharmacology, 2:56-62. Most current pharmacological therapies for schizophrenia and related disorders target these pathways. The clinical results obtained with this generation of atypical antipsychotic drugs fail to indicate a substantial increase in potency over typical antipsychotics, but have gained popularity because this new generation of remedies reduces the discomfort caused by side effects of more selective dopamine receptor antagonists. However, the affinity of these drugs for multiple receptors leaves much uncertainty over the precise targets that could better ameliorate the symptoms of schizophrenia.

A number of animal models of schizophrenia induced by treatment with phencyclidine (PCP) or amphetamine display increases in locomotion and these behaviors have been correlated with the symptoms of schizophrenia (Corbett et al. (1995) Psychopharmacology (Ber1) 120:67-74; Moghaddam et al. (1998). Science 281:1349-1352). Locomotor activity in animals is primarily regulated by monoaminergic neurotransmitter systems, particularly dopamine and serotonin (Akunne et al. (1992) Neurochem. Res. 17:261-264; Miller et al. (1996). Brain Res. Bull. 40:57-62; Rothman (1994) Neurotoxicol. Teratol. 16:343-353). However, the alterations in behavior caused by PCP and amphetamine treatment are extensive. These treatments also cause neuronal loss, atrophy, neuroglial proliferation, and some cavitation in the ventral hippocampus while sparing the dorsal hippocampus, thus creating confounding obstacles in the assessment of any potential therapeutic treatment

Some animal models of schizophrenia can be induced with other pharmacological agents, such as dopamine receptor agonists or antagonists. However, many of these drugs can cause sedation, thus highlighting a problem that requires careful dosing of animals to achieve a reproducible level of impairment, yet maintain consciousness and responsive capacity so that the animal can react to the therapy being tested.

Prepulse inhibition (PPI) is a phenomenon in which a weak prestimulus or prepulse suppresses the response to a subsequent startling stimulus, and has been found to be impaired in schizophrenia and schizotypal personality disorders. Until recently, the rat has been the predominant species used for studying the neurobiology of PPI. However, many of these rat models require extensive lesioning of brain regions, such as those induced by PCP treatment. Because these lesions can be debilitating in a confounding fashion, mutant mouse models of impaired PPI are gaining in popularity. See Geyer et al, 2002, Molecular Psychiatry 7:1039-1053. However, PPI is just one of several measures of impairment, and does not constitute a sufficiently thorough assessment of drug efficacy for treating schizophrenia.

Despite the increasing number of animal models of different aspects of schizophrenia and related disorders, a need exists for a precise lesion that maintains brain architecture and neuronal circuitry while disrupting only those pathways impacting on these disorders. An ideal animal model would incorporate multiple aspects of schizophrenia that could be easily tested. Given the devastating consequences of schizophrenia, such a model would fulfill a need for testing and developing novel therapies in a rapid and efficient manner.

There is also a need to evaluate existing therapies in appropriate animal models. Although new generation anti-psychotics are increasingly replacing conventional agents such as chlorpromazine and haloperidol in some countries, many issues about these compounds need to be clarified. See Leucht et al. (2003) Lancet, 362:1581-9. Of all the new generation drugs, only clozapine has proven better than low-potency conventional drugs in patients with schizophrenia that is resistant to treatment. Whether the new antipsychotics have an effect on primary negative symptoms or only on secondary negative symptoms are debatable. Although results of a meta-analysis showed that use of the new drugs led to a modest, but significant, reduction of schizophrenic relapses, the role of improved compliance in the analysis was unclear.

The need for appropriate animal models is also apparent in the scientific literature concerned with the long-term effects of treatment for schizophrenia and schizoaffective disorders. See Baethge (2003) Pharmacopsychiatry, 36:45-56. Conclusive data on long-term therapies is lacking, and even studies using the most sophisticated methodologies in the field are very limited. Thus, a need exists for an animal model that can demonstrate within 1-2 years or less potential long-term effects of a drug.

The recent discovery of a disruption of the neuronal PAS3 (Npas3) gene in a family affected with schizophrenia allowed identification of a novel pathway involved in the pathogenesis of the disorder. See Kamnasara et al, (2003) J. Med. Genet. 40:325-332. The mouse Npas3 gene was subsequently found to be expressed in a pattern consistent with its putative role in schizophrenia (see Brunskill et al. (1999) Mech. Of Dev. (88)2: 237-241) and is the basis for the mouse model in the present invention and methods for its use described herein.

### BRIEF DESCRIPTION OF THE INVENTION

The invention relates to a transgenic mouse having a genome that comprises a mutation of an endogenous Npas3 gene, wherein the Npas3 mutation causes a disruption that inactivates the gene, wherein a homozygous transgenic Npas3 mutant mouse does not produce a fully functional NPAS3 protein. The invention also relates to a cell isolated from the mouse.

The invention further relates to a method for determining the effectiveness of a biologically active agent in a transgenic mouse, comprising the steps of disrupting an endogenous Npas3 gene in the transgenic mouse wherein the disruption inactivates the gene, administering to the mouse the biologically active agent, and assessing for a change in a phenotype of the mouse.

The invention further relates to a method for determining the effectiveness of a biologically active agent in at least one cell of a transgenic mouse, comprising the steps of disrupting at least one allele of an endogenous Npas3 gene in the transgenic mouse wherein the disruption inactivates the gene, isolating at least one cell from the transgenic mouse, administering to the isolated cell the biologically active agent, and detecting a biochemical change in the isolated cell.

The invention further relates to a method for determining the effectiveness of a biologically active agent in a cell line derived from a transgenic mouse, comprising the steps of disrupting at least one allele of the endogenous Npas3 gene in the transgenic mouse wherein the disruption inactivates the gene, isolating at least one cell from the transgenic mouse, deriving an immortalized cell line from the isolated cell, amplifying the cells of the cell line, administering at least one biologically active agent to the cells of the cell line, and detecting a biochemical change in the cells of the cell line.

### BRIEF DESCRIPTION OF THE SEQUENCE LISTINGS

SEQ ID NO:1 shows the nucleotide sequence of a genomic DNA fragment of the mouse Npas3 gene (mus musculus).

### BRIEF DESCRIPTION OF THE DRAWINGS

FIG. 1 shows schematic maps of an Npas3 Endogenous Locus, a Targeting Vector, and a Targeted Locus.

FIG. 2 shows a Southern blot analysis of DNA from targeted (C2) and untargeted (C3) embryonic stem cell clones.

FIG. 3 shows genotyping PCR analysis from tail DNA samples of Npas3+/+, Npas3^{+/-} and Npas3−/− mice.

FIG. 4 shows a Northern blot analysis of Npas3 gene expression using poly-A RNA isolated from the brains of Npas3+/+ control and Npas3−/− mice.

FIG. 5 shows a photograph representative of Npas3+/+ control (left) and Npas3−/− mice (right) two days postnatal.

FIG. 6 shows a representative 40 day growth curve for Npas3+/+ control and Npas−/− mice.

FIG. 7 shows gross morphological appearance of brains from an Npas3+/+ control mouse (left panel, C) and an Npas3−/− mouse (right panel, E).

FIG. 8 shows a hematoxylin and eosin-stained coronal section from a brain of an Npas3+/+ control mouse (upper panel) and an Npas3−/− mouse (lower panel).

FIG. 9 shows a significant alteration in the cerebellum folia of an Npas3−/− mouse brain (right panel, d) compared to an Npas3+/+ control brain (left panel, f).

FIG. 10 shows a histological analysis of brain sections from Npas3+/+ control (left panel, e) and Npas−/− (right panel, f) mice.

FIG. 11 shows a magnetic resonance image (MRI) of Npas3+/+ control (left panel, g) and Npas−/− (right panel, h) mice.

FIG. 12 shows a Tail Suspension Test of an Npas3+/+ control mouse (left) and an Npas3−/− mouse (right).

FIG. 13 shows representative Footprint Test patterns of an Npas3+/+ (wild type or WT) control mouse (left side) and an Npas3−/− mouse (right side).

FIG. 14 shows an analysis of Stride length, in Npas3+/+ control (white bars) and Npas+/+ (black bars) mice.

FIG. 15 shows an analysis of Hind paw Base Width in Npas3+/+ control (white bars) and Npas−/− (black bars) mice.

FIG. 16 shows an analysis of Forepaw Base Width in Npas3+/+ control (white bars) and Npas−/− (black bars) mice.

FIG. 17 shows an analysis of Forepaw Hind paw Overlap in Npas3+/+ control (white bars) and Npas−/− (black bars) mice.

FIG. 18 shows a comparison of times for Beam-walking Tests of Npas3+/+ or wild type (WIT) control (white bars) and Npas−/− (black bars) mice.

FIG. 19 shows a comparison of Footslips for Beam-walking Tests of Npas3+/+ or wild type (WT) control (white bars) and Npas−/− (black bars) mice.

FIG. 20 shows horizontal activity counts of a Locomotor Activity Test following administration (arrow) of methamphetamine in Npas−/− (triangles) and Npas3+/+ control (squares) mice.

FIG. 21 shows the % change in horizontal activity of a Locomoter Activity Test following administration of methamphetamine, as shown in FIG. 20, in Npas3−/− (solid circles) and Npas3+/+ control (open circles) mice.

FIG. 22 shows horizontal activity counts of a Locomotor Activity Test following administration (arrow) of saline in Npas−/− (triangles) and Npas3+/+ control (squares) mice.

FIG. 23 shows the % change in horizontal activity of a Locomotor Activity Test following administration of saline, as shown in FIG. 22, in Npas3−/− (solid circles) and Npas3+/+ control (open circles) mice.

FIG. 24 shows horizontal activity counts of a Locomotor Activity Test mice following administration (arrow) of 0.3 mg/kg haloperidol in Npas3+/+control (open circles) and Npas3−/− (solid circles) mice.

FIG. 25 shows horizontal activity counts of a Locomotor Activity Test mice following administration (arrow) of 1.0 mg/kg haloperidol in Npas3+/+ control (open circles) and Npas3−/− (solid circles) mice.

FIG. 26 shows horizontal activity counts of a Locomoter Activity Test following administration (arrow) of quinpirole in Npas3+/+ control (open circles) and Npas−/− (solid circles) mice.

FIG. 27 shows horizontal activity counts of a Locomoter Activity Test following administration (arrow) of clozapine in Npas3+/+ control (open circles) and Npas3−/− (solid circles) mice.

FIG. 28 shows horizontal activity counts of a Locomoter Activity Test following administration (arrow) of MK-801 in Npas3+/+ (wild) control (open circles) and Npas−/− (solid circles) mice.

FIG. 29 shows an analysis of NMDA receptor density in Npas+/+and Npas−/− mice measured in an MK-801 binding assay.

FIG. 30 shows the baseline startle response and Prepulse Inhibition (PPI) test response of Npas+l+and Npas−/− mice.

FIG. 31 shows the percent change (Vmax) from baseline startle response in a PPI test of Npas+/+(WT) and Npas−/− mice.

FIG. 32 shows the results of an anxiety assessment using a Zero Maze Test, including time spent in open areas, stretch-attends movements, and number of open area entries.

FIG. 33 shows an analysis of time spent with a novel object by Npas3+/+and Npas−/− mice.

FIG. 34 shows comparison of nesting behavior by Npas3+/+ (upper panel) and Npas−/− (lower panel) mice.

### DETAILED DESCRIPTION OF THE INVENTION

### 1.Definitions:

As used herein the term “Npas3” refers to a mammalian gene expressed in Neuronal and other tissues, and belonging to a family of basic helix-loop-helix (bHLH) transcription factors containing a PAS domain (so named for homology with drosophila genes known as Period, Aryl hydrocarbon receptor, and Single minded).

As used herein, the terms “Npas3 gene” and “Npas3 locus” are used interchangeably in reference to the nucleotide sequences encoding the mammalian gene that produces the NPAS3 protein product.

As used herein, the terms “Npas3-deficient”, “Npas3−/−”, and “Npas3-mutant” are used interchangeably in reference to a transgenic mouse or transgenic cell with a gene-targeted mutation of the Npas3 gene locus such that expression of the Npas3 gene is disrupted.

As used herein, the term “homozygous” refers to a mouse or a cell with two identical alleles of any genomic DNA nucleotide sequence of interest. The term “heterozygous” refers to a mouse or a cell with at least one differing nucleotide between two alleles for any genomic DNA nucleotide sequence of interest

As used herein, the terms “Npas3+/+”, “Npas3+/+ control”, and “wild-type” are used interchangeably in reference to a mouse with intact endogenous Npas3 gene loci.

As used herein, the term “transcription factor” refers to a nuclear protein that modulates expression of a target gene by binding its cognate recognition sequences within the regulatory region of the target gene and influencing the rate of transcription of the gene.

As used herein, the terms “Neomycin” and “neomycin-resistance” refer to nucleotide sequences used to confer resistance to the antibiotic neomycin or its pharmacological analog, G418.

As used herein, the terms “exon” or “exonic” refer to a distinct region of nucleotide sequence within a gene that encodes a region of a proprotein (an intermediate protein product) or a final protein product of the gene.

As used herein, the terms “intron” or “intronic” refer to a region of nucleotide sequence contained within a eukaryotic gene that is excised from an mRNA transcript of the gene, and thus do not encode a portion of a proprotein or a final protein product of the gene.

As used herein, the term “129 genomic library” refers to a collection of genomic DNA fragments obtained from a mouse of the strain known as 129.

As used herein, the term “C57B16” refers to a mouse or mice of the C57-Black-6 genetic strain of mus musculus.

As used herein, the term “chimeric” refers to a mouse or mice produced by injecting cells derived from one source into a blastocyst derived from a second source.

As used herein, the terms “HindIII”, “BamHI”, “XbaI”, “XhoI”, and “NotI” refer to specific restriction endonucleases that recognize and cleave specific short combinations of double-stranded DNA nucleotide sequence.

As used herein, the terms “immortalized”, and “transformed” are used interchangeably to refer to a cell or cell line with cancer-like properties that allow indefinite rounds of cell division.

### 2. Detailed Description of the Npas3−/− Mouse:

The present invention is a transgenic mouse with a gene-targeted mutation of an endogenous Npas3 gene that results in disruption of the Npas3 gene. The Npas3−/− mouse is a model of schizophrenia, with impairments to locomotor activity that are shared with other neurological disorders, such as Parkinson's Disease. Other related neurological disorders include Obsessive-Compulsive Disorder, Tourette's Syndrome, and bipolar disorders. The invention is also a method for testing the efficacy of a biologically active agent in the treatment of schizophrenia and related neurological disorders. The Npas3−/− mouse can be used to test a biologically active agent, such as a pharmaceutical compound, a small molecule, or recombinant protein, by administering the agent and testing for changes in a behavioral or biochemical phenotype of the Npas3−/− mouse. Furthermore, the invention relates to a cell or cell line derived from the Npas−/− mouse. The invention also relates to a method for using the cell or cell line to screen biologically active agents to determine if the biologically active agent can alter the biochemical phenotype of the cell or cell lines derived from the Npas3−/− mouse. Npas3−/− cells can be used to test the efficacy of biologically active agents, such as pharmaceutical compounds, small molecules, or recombinant proteins, and are particularly useful in high throughput screening of small molecule or compound libraries.

The NPAS3 protein product of the Npas3 gene is a member of the basic helix-loop-helix (bHLH) PAS family of transcription regulators and is expressed throughout the developing neuroepithelium. The members of the bHLH family are a group of related proteins that are involved in a number of biological and physiological processes such as the regulation of myogenesis (MyoD/Mef), neurogenesis (NeuroD), toxin metabolism (ARNT/Ahr) and circadian rhythms (clock/period). These proteins contain a basic region that is involved in DNA binding and a helix-loop-helix region that is responsible for protein dimerization. A subset of the bHLH family of genes encodes a 200-300 block of amino acid similarity known as the PAS domain. The PAS domain consists of two degenerate 50 amino acid direct repeats. These repeats serve to mediate protein dimerization specificity between other PAS proteins, small molecule binding, and interactions with non-PAS proteins. Disruptions or mutations in any of these protein domains can alter the full function of NPAS3 protein, and can be implicated in neurological disorders associated with Npas3 gene expression.

Analysis of Npas3 gene expression patterns suggests that this gene plays a broad role in neurogenesis. See Brunskill et al. (1999) Mechanisms of Development 88:237-241. Although the specific pathophysiological processes and etiological factors that alter the neurochemical signaling pathways in schizophrenia patients have proven elusive, the recent identification of schizophrenic patients with a deletion of the Npas3 gene has provided insight into the possible genetic pathways that underlie this profound neuropsychiatric disturbance.

In the present invention, Npas3-deficient mice are generated using an Npas3 Targeting Vector. In order to build the Targeting Vector, genomic clones containing the bHLH exon of the Npas3 gene are isolated from a mouse 129 genomic library. The 20 kilobase (Kb) nucleotide sequence of the genomic clones is shown in SEQ ID NO: 1. Nucleotide sequences for 5′ and 3′ targeting arms are amplified from these genomic clones with high-fidelity DNA polymerase in two polymerase chain reactions (PCR).

The 5′ targeting arm of the Targeting Vector is generated using oligonucleotides 5′-TCAAAGCTTTCACAGTCTTTGCTGATGATT-3′ and 5′-GTAAAGCTTAGGCAAAGATCTTAGACCAGA-3′. These sequences include HindIII restriction sites (underlined). Amplified products are isolated, digested with HindIII, purified and cloned into the HindIII site of a pNTKVNeo vector (Stratagene, La Jolla, Calif.).

The 3′ targeting arm of the Targeting Vector is generated using oligonucleotides 5′-GTAGGATCCTCTCTGGAATGAAATGTTCACCAGC-3′ and 5′-GCAGGATCCATGCATGCATGGCTCACATGG-3′. These sequences include BamHI restriction sites (underlined). Amplified products are isolated, digested with BamHI, purified and cloned into the BamHI site of pNTKVNeo.

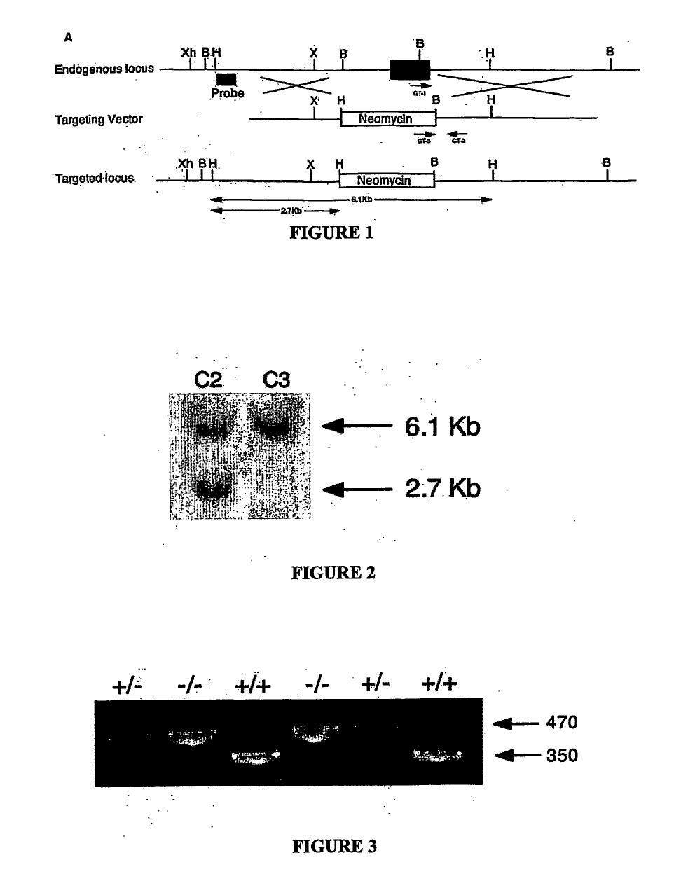

A targeting construct map labeled as Targeting Vector is shown in FIG. 1. The backbone of the targeting construct is a fragment of mouse genomic DNA comprising two intronic regions flanking an exon that encodes a bHLH domain of NPAS3 protein. As illustrated in the Endogenous Locus map, the general location of the Npas3 bHLH exon is indicated by a black box superimposed on a vertical line that represents the fragment of the nucleotide sequences encoding the wild type Npas3 gene.

Homologous recombination of the general regions indicated by two X's between the Endogenous Locus and Targeting Vector results in the replacement of the bHLH exon with a Neomycin-resistance cassette. The product of this recombination event is depicted as the Targeted Locus. The maps also provide an overview of a strategy for detecting positive recombinant alleles. Restriction enzyme sites are indicated by the letters B (BamHI), H (HindIII), X (Xbal), and Xho (XhoI). Integration of the neomycin cassette embedded in the Targeting Vector results in the insertion of a HindIII restriction endonuclease recognition site in the Targeted Locus. Expected fragment lengths produced by a HindIII digestion are indicated by double-headed arrows for the wild type (6.1 Kb) and targeted alleles (2.7 Kb). The location of oligonucleotides used for PCR genotyping of Npas3-mutant mice is shown as single headed arrows.

The fully assembled Targeting Vector is linearized with NotI and electroporated into KG-1 embryonic stem (ES) cells. Integration of the neomycin cassette embedded in the Targeting Vector inserts a new HindIII site into the targeted allele, generating a diagnostic 2.7 Kb HindIII fragment. ES cell DNA is isolated from candidate clones and digested with HindIII. Genotypes of clonal populations of ES cells are determined using a ˜500 base pair (bp) Npas3 probe (Probe) generated from Npas3 sequences 5′ of the sequences used in the Targeting Vector. Sequences encoding the Probe are indicated in FIG. 1 by a black box below the Endogenous Locus map. The Probe is amplified by PCR using the oligonucleotides with the nucleotide sequences 5′-AAGGTTTCCTGCACATAC-3′ and 5′-AATCATCAGCAAAGACTG-3′. Correctly targeted G418-resistant clones are identified by Southern blotting of the HindIII-digested DNA that demonstrates the presence of 6.1 and 2.7 Kb bands, as illustrated by clone C2 in FIG. 2, while wild type (untargeted) clones have only a 6.1 Kb band, similar to clone C3.

Correctly targeted ES cells are injected into C57B1/6 recipient blastocysts to obtain chimeric progeny. Chimeric mice are selected on the basis of coat color, and are bred with C57B1/6 mice to obtain germline transmission of the targeted allele. Heterozygous animals are intercrossed to obtain homozygous Npas3−/− mice. Genotypes of animals are confirmed by PCR of tail DNA using the oligonucleotides GT-1 (5′-TCCTGACTA GGGGAGGAGTAGAAG-3′), GT-2 (5′-CACATTAGCTCTTACCTATGAGCC-3′); and GT-3: (5′-ACCTGAGGATGGAAGGCCCTCCAC-3′). The non-targeted wild type allele generates a 350 bp PCR product and the targeted allele generates a 470 bp PCR product. Homozygous offspring are born at the expected frequency of 25%, with analysis of genomic DNA from a representative group of animals illustrated in FIG. 3.

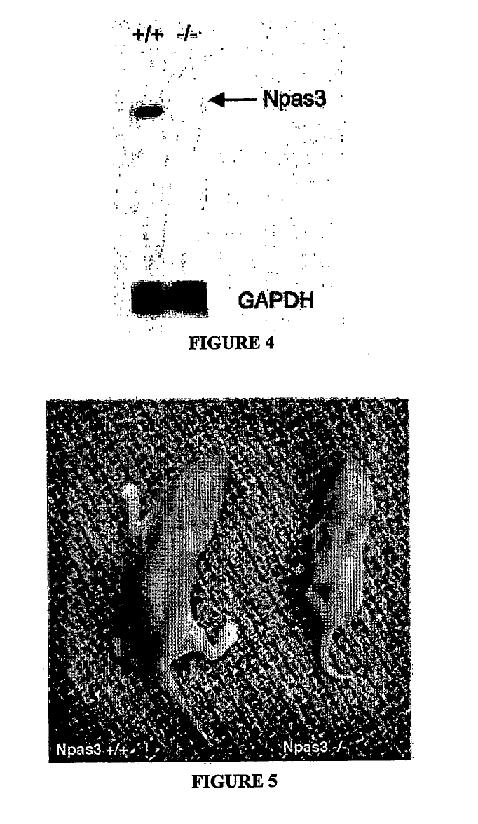

Disruption of Npas3 gene expression is verified by Northern blot analysis. Total RNA is prepared using RNAzol B (TelTest, Friendswood, Tex.) from brains of 8-week-old mice. Poly-A RNA is isolated using an Oligotex mRNA kit following manufacturer's protocol (Qiagen, Calif.). One microgram of poly-A RNA of each genotype is electrophoresed on a 1.2% agarose/1.1% formaldehyde gel and transferred to nitrocellulose membrane. The membrane is hybridized with labeled sequences specific for the bHLH exon of Npas3. As illustrated in FIG. 4, no Npas3 mRNA is detected in Npas3−/− mice. The membrane is then stripped and probed for mouse glyceraldehyde 3-phosphate dehydrogenase (GAPDH) gene expression. Analysis of GAPDH expression is well-known to those skilled in the art of analyzing gene expression, and verifies that the amount and integrity of the RNA loaded in each lane are similar.

Alternative methods for generating Npas3-mutant mice can also be envisioned, and can be used in the practice of the present invention, as well. This can include heterozygous Npas3^{+/-} mice, which can produce lower levels of Npas3 MRNA transcripts and NPAS3 protein. Another embodiment is an Npas3-mutant mouse generated using Cre-lox or other conditional gene deletion “knockout” technology, which is well known to those skilled in the art of gene-targeting mice. Another example of a conditional knockout can be made using FRT-Flpase technology, also well know to those skilled in the art of gene-targeting mice. Other embodiments include a wide variety of mice with mutations that impair NPAS3 protein function, but do not cause a null-mutation of Npas3. Any nucleotide in an exonic region of the Npas3 gene can be mutated or deleted in order to produce a mouse with impaired NPAS3 protein function, i.e., a mouse that retains Npas3 gene expression, but does not have fully functional NPAS3. Such mice can be generated with a targeting construct containing a site-specific mutation, or as a “knock-in”, in which a mutated sequence is targeted to an acceptor site, such as one generated in a conventional gene-targeting strategy.

The phenotype of schizophrenia can also be enhanced by crossing an Npas3−/− mouse with a mouse that has a mutation of another gene that causes similar or partial neural deficit(s), such as those observed in the Npas3−/− mice. Genes that compensate for Npas3 function are particularly well-suited for this mode of practice of the invention. Such genes include, but are not limited to, neuroregulin, Npas1, and reelin. Combinatorial mutants of any or all of these genes can enhance the neural dysfunction in the mice, resulting in a more profoundly impaired model of schizophrenia. Such mutations can completely ablate gene expression of one of the combinatorial genes, such as is seen in the homozygous Npas3−/− mice, or may be heterozygous for one or more of the combinatorial genes. In addition, the Npas3−/− mice could be crossed with mice harboring partial mutants of genes such as neuroregulin, Npas1, and reelin, wherein, a mutation causes expression of a protein with partial function. Such a mouse would then have no Npas protein expression, combined with impaired protein function of the combinatorial gene contributing to the schizophrenic phenotype.

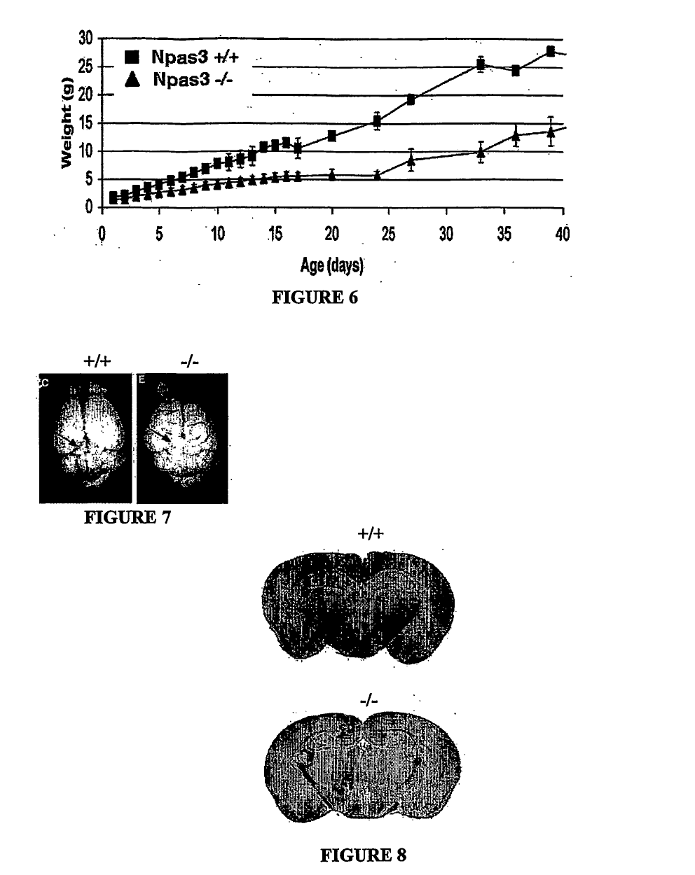

Although homozygous Npas3−/− mice initially appear to develop normally at birth, by day 2 the mutant mice can be distinguished from Npas3+/+ mice by a reduction in size, shown in FIG. 5. The Npas3−/− mice (triangles) are smaller than Npas3+/+control mice (squares) throughout postnatal development and remain 20-35% smaller as adults, as shown in a 40-day growth curve, FIG. 6.

Potential neuropathological changes associated with Npas3−/− mice are assessed in mutant and wild-type brains. For histology, mice are perfused intracardially with 50 ml of 4% paraformaldehyde in phosphate-buffered saline (PBS). The brain is quickly removed and post-fixed overnight at 4° C. in 4% paraformaldehyde. The tissue is embedded in paraffin, cut into 5 mm-thick sections, and stained with hematoxylin and eosin. At the gross morphological level, shown in FIG. 7, the brains from Npas3−/ mice (right panel) appear normal, although there is a slight reduction in size of the posterior region of the neocortex (indicated by arrow for each brain) compared to that of Npas3+/+ controls (left panel).

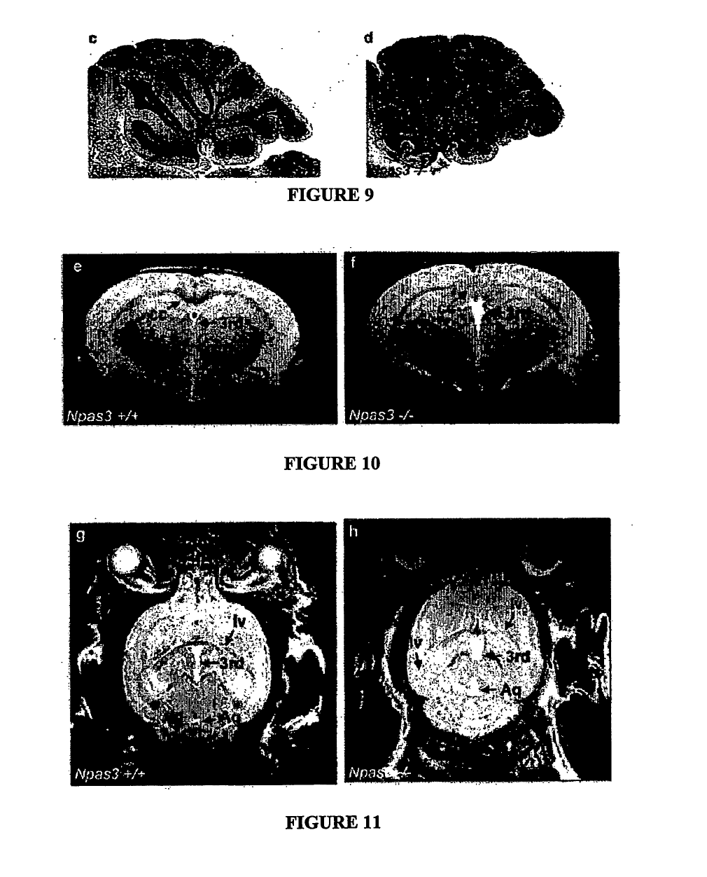

Neuroimaging has revealed characteristic abnormalities in the brains of schizophrenic patients (see Shenton et al., Schizophrenia Res 49(1), Apr. 15, 2001). Similar neuropathological alterations are found in brains of Npas3−/− mice. Histological examination of coronal sections from the brains of Npas3−/− mice reveals abnormalities in the development of cortical-limbic as well as dorsal thalamic regions. The cingulate cortex (cg) of Npas3−/− mice is considerably enlarged and this enlargement coincides with a reduction in the size of the hippocampus. FIG. 8 shows this expansion of the cingulate cortex (cg) that is typical of Npas3−/− mice (lower panel) compared to Npas3+/+ controls (upper panel) as well as the alterations in the size and shape of the hippocampus (darker hematoxylin-staining cells forming “horn” shapes under the cingulate cortex). Developmental abnormalities of the cerebellum are also found, including a significant alteration in the folia of Npas3−/− (right panel) compared to Npas3+/+ controls (left panel), as shown in FIG. 9. As shown in FIG. 10, decreased numbers of commissural fibers in the corpus callosum (cc) above the third ventricle (3^{rd}) are also revealed in Npas3−/− mice (right panel), compared with in Npas3+/+ mice, (left panel). Magnetic resonance imaging (MRI), shown in FIG. 11, confirmed the histological findings and also revealed enlargement of the posterior lateral (left Iv), third (3^{rd}) and fourth (right Iv) ventricles, as well as an enlargement of the aqueduct of Sylvius (Aq)in Npas3−/− mice (right panel), compared with in Npas3+/+ mice (left panel),

### 3. Behavioral Analysis of the Npas3−/− Mouse:

Morphological alterations in several brain regions suggest that Npas3−/− mice would have behavioral abnormalities. The following tests are used to determine the baseline neurological status of the Npas3−/− mice: Tail Suspension Test, Footprint Test, Beam-walking Test, and Locomotor Activity Tests. These tests are also used in practicing the methods for testing biologically active agents in Npas3−/− mice, and embodiments of these methods will refer to the test protocols. Following the description of each of these test protocols, the baseline response of Npas3−/− mice is given. As can be appreciated from the results of these tests, overt motor behavioral abnormalities can be evaluated in Npas3−/− mice by 3 weeks of age.

Tail Suspension Test. The test mouse is lifted upwards by the tail, such that its feet lose contact with a supporting surface, and the mouse is hanging freely by its tail. The characteristic response of a wild-type mouse is to extend its limbs and struggle. In general, an immobilized feet-clasping posture is associated with mouse models of neurological disorders affecting locomotor activity. Qualitative scores of positive (normal) or negative (impaired) responses are recorded for each mouse, with n=12.

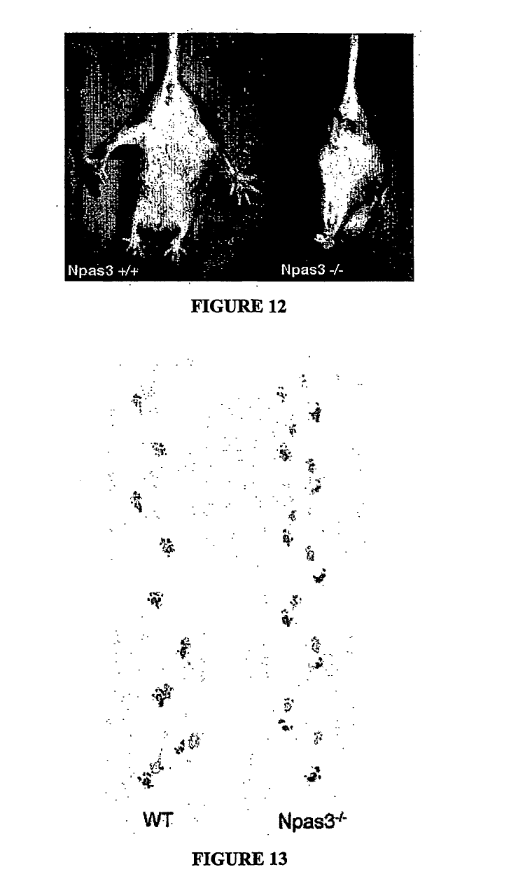

Results of baseline Tail Suspension Test in Npas3−/− mice, are shown in FIG. 12. When lifted up by their tails, all 12 Npas3−/− mice reflexively contract their limbs and remain immobile, whereas all 12 Npas3+/+mice extend their limbs and struggle. The left panel of shows a typical Npas3+/+ control mouse displaying a characteristic positive response of splayed hind limbs, while the right panel shows a typical Npas3−/− mouse in a negative feet-clasping posture. This result is consistently evident in Npas3−/− mice as young as 3-4 weeks of age.

Footprint Test. The hind feet and forefeet of test mice are coated with purple and orange nontoxic paints, respectively. The animals are then allowed to walk along a 50-cm-long, 10-cm-wide runway (with 10-cm-high walls) into an enclosed box. All mice receive three training runs and are then tested. A fresh sheet of white paper is placed on the floor of the runway for each test run. The footprint patterns are analyzed for four step parameters: stride length, hind paw-base width, forepaw-base width and forepaw/hind paw overlap. Each measurement represents the average over three consecutive steps during a test run. Gait parameters are statistically analyzed using a Student's t-test. All Footprint Tests are performed on mice at 7-9 weeks of age, with n=12. All values are expressed as mean±SEM. Statistical analyses are performed using appropriate analysis of variance (ANOVA) followed by adapted post hoc comparisons.

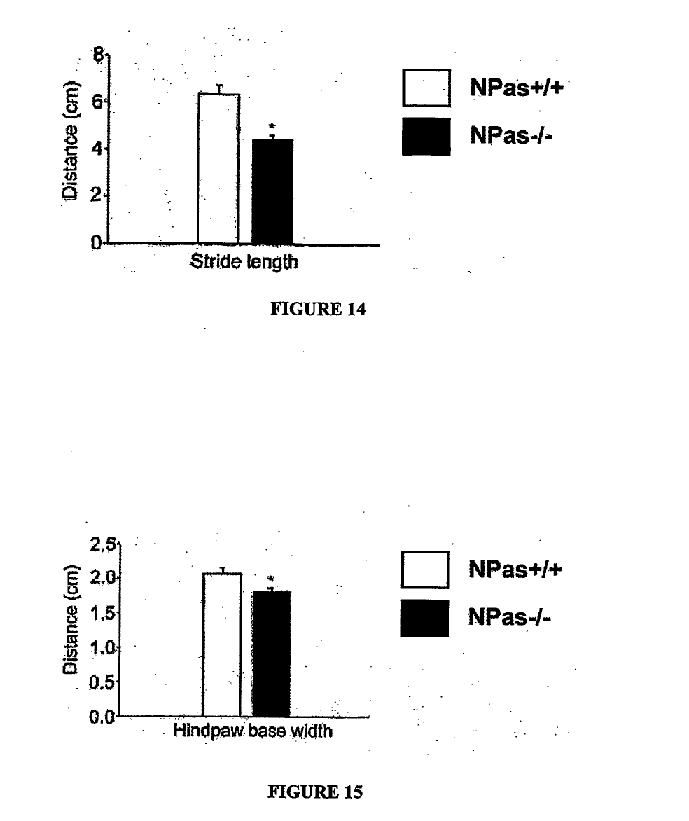

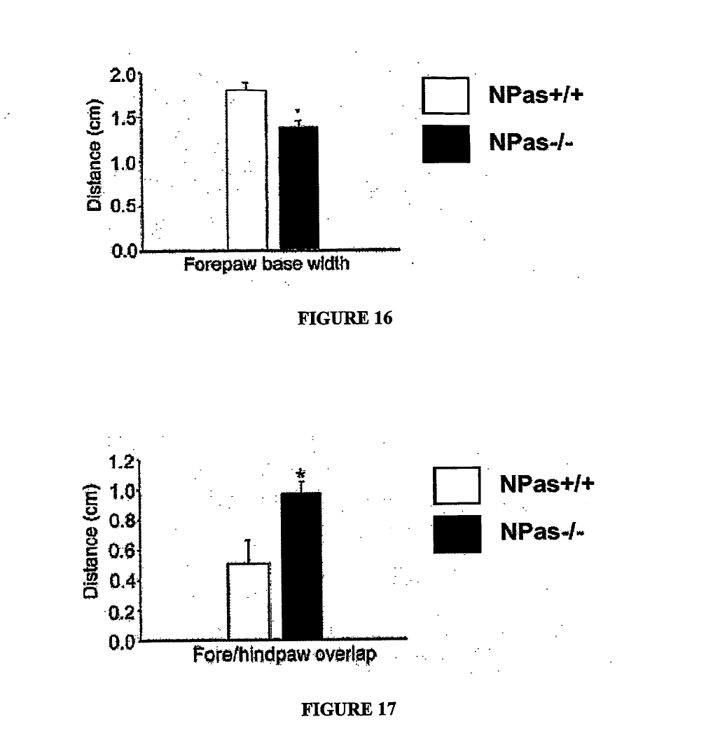

Results of baseline Footprint Test in Npas3−/− mice, FIG. 13, show that Npas3−/− mice (right panel) have a parkinsonian gait that consists of unevenly spaced shorter strides, staggering movements and a gait that lacks a normal, uniform, alternating step pattern found with Npas3+/+control mice (left panel). Qualitatively, the footprint patterns clearly differ, showing that the Npas3−/− mice display irregular, shorter strides and an uneven step pattern. Quantitative analyses of stride length (FIG. 14), hind paw-base width (FIG. 15), forepaw-base width (FIG. 16) and forepaw/hind paw overlap (FIG. 17) in the walking footprint patterns produced by Npas3+/+(white bars) and Npas3−/− (black bars) mice demonstrate significant differences in all four parameters.

Beam-walking Test. Motor coordination and balance are assessed by measuring the ability of test mice to traverse a graded series of narrow square or round beams and reach an enclosed safety platform. The beams are interchangeable strips of wood 1-meter in length. Two beams are square, and 3 are round, similar to dowel rods. The beams have square cross-section thicknesses of either 25- or 12-mm, or have diameters of 28-, 17-, or 11-mm. When assembled for use, a beam is placed horizontally 50 cm above a laboratory bench surface with one end mounted on a narrow support (“starting end”) and the other end attached to an enclosed box (20 cm square) into which the mouse can escape. During training, mice are placed at the starting end of the 25-mm square beam and trained over 3 days (4 trials per day) to traverse the beam to the enclosed box. Once the mice are trained (able to traverse the 25-mm square beam in less than 20 seconds) they receive two consecutive test trials on each of the square beams and each of the round beams, in each case progressing from the widest to the narrowest beam. Mice are allowed up to 60 seconds to traverse each beam. The latency to traverse each beam (“Time”) and the number of times the hind feet slip off each beam (“Footslips”) is recorded for each trial. Analysis of each measure is based on the mean scores of the two trials for each beam. All Beam-walking Tests are performed on mice at 7-9 weeks of age, with n=12. All values are expressed as mean±SEM. Statistical analyses are performed using appropriate analysis of variance (ANOVA) followed by adapted post hoc comparisons.

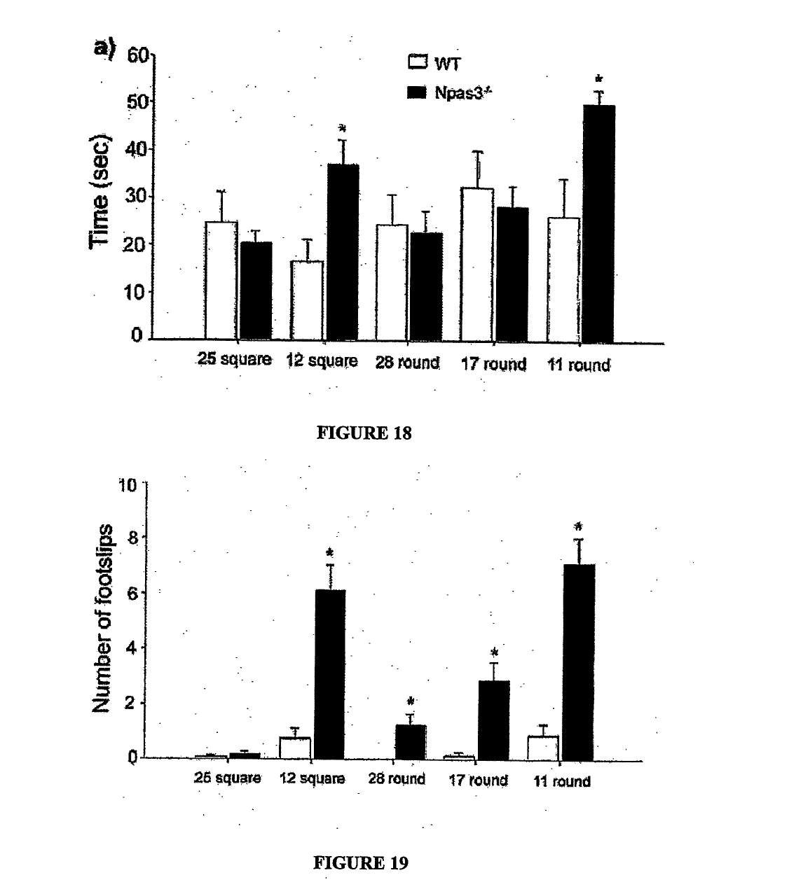

Results of Beam-walking Test in Npas3−/− mice show that Npas3−/− mice (black bars) are significantly impaired in fine motor coordination and balance as assessed by beam walking on narrow bridges compared to Npas3+/+ control mice (white bars). The Time and Number of Footslips are shown in FIGS. 18 and 19, respectively. Asterisks indicate significant differences between groups of Npas3+/+and Npas3−/− mice (* p<0.05).

Locomotor Activity Tests. For all locomotor experiments, activity is measured in a 41×41×30 cm Omnitech Digiscan activity monitor equipped with 16 pairs of photodetector-LED beams along the x and y axis. (Accuscan Electronics, Columbus, Ohio). Test mice are acclimated to the chamber for 15 minutes or 60 minutes, depending on the test. After the acclimation period, Baseline Locomoter Activity Test results are recorded for each test mouse. Subsequently, locomotor activity is tested following pharmacological challenges of three neurotransmitter pathways involved in schizophrenia and other neurological disorders. The pathways and the drugs used to challenge each include:

### Dopamine Pathway Locomoter Activity Tests:

a. Methamphetamine (a dopamine pathway agonist) 1.0 mg/kg in the form of D-methamphetamine-HCL (Sigma; St. Louis, Mo.). Methamphetamine is a strong dopamine agonist that will activate D1 and D2 receptor signaling.

b. Haloperidol (a dopamine pathway antagonist) 0.3 mg/kg or 1.0 mg/kg (Tocris; Ellisville, Mo.). Haloperidol is an antipsychotic drug and a potent D2-receptor antagonist with relatively high specificity.

c. Quinpirole (a dopamine pathway antagonist), 1.0 mg/kg (Sigma; St. Louis, Mo.). Quinpirole is a potent D2-receptor agonist.

### Serotonin Pathway Locomotor Activity Test:

The test drug is 0.3 mg/kg clozapine (Sigma; St. Louis, Mo.). An atypical neuroleptic agent, clozapine, which has a high affinity for serotonin 5-HT₂ and 5-HT₆ receptors, is administered to determine the involvement of serotonergic pathways. Although clozapine can also antagonize both D1 and D2-like receptors, ten times higher concentrations are necessary to inhibit dopamine-dependent hyperactivity

### Glutamate Pathway Locomotor Activity Test:

The test drugs are 1.0 mg/kg MK-801 or 0.3 mg/kg MK-801 (ICN Biomedicals Inc.; Aurora, Ohio). MK-801 is a noncompetitive NMDA receptor antagonist.

Each drug is administered in a subcutaneous injection volume of 5 mL/kg body weight. Post-challenge activity is measured for three hours. Horizontal activity counts (scored by visual observance or breaking a light beam) and total distance are recorded during the pre- and post-challenge periods at 3-minute intervals with VersaMax software. All Locomoter Activity Tests are performed on mice at 7-9 weeks of age, with n=12. All values are expressed as mean±SEM. Statistical analyses are performed using appropriate analysis of variance (ANOVA) followed by adapted post hoc comparisons.

### Results of Locomotor Activity Tests in Npas3−/− Mice:

Baseline Locomoter Activity Test: Typically, wild-type mice exhibit an increase in motor activity during habituation to a new environment Accordingly, Npas3+/+control mice demonstrate a substantial increase in horizontal locomotor activity (˜900%) over pre-challenge activity. However, only a negligible increase of locomotor activity is seen in the Npas3−/− mutants during the Baseline Locomotor Activity Test

### Dopamine Pathway Locomotor Activity Tests

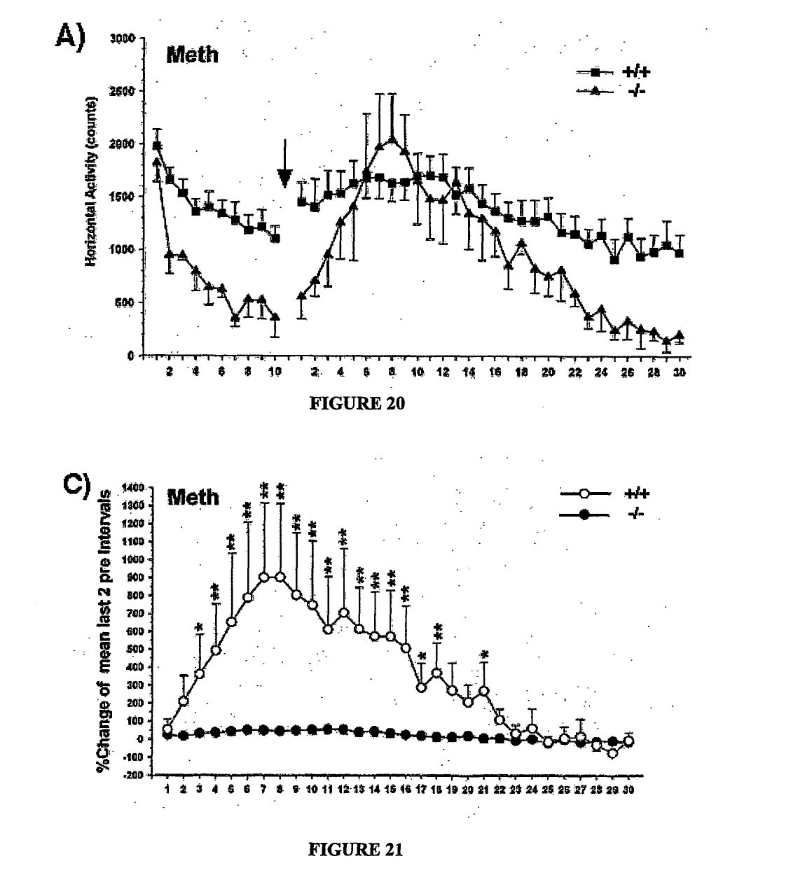

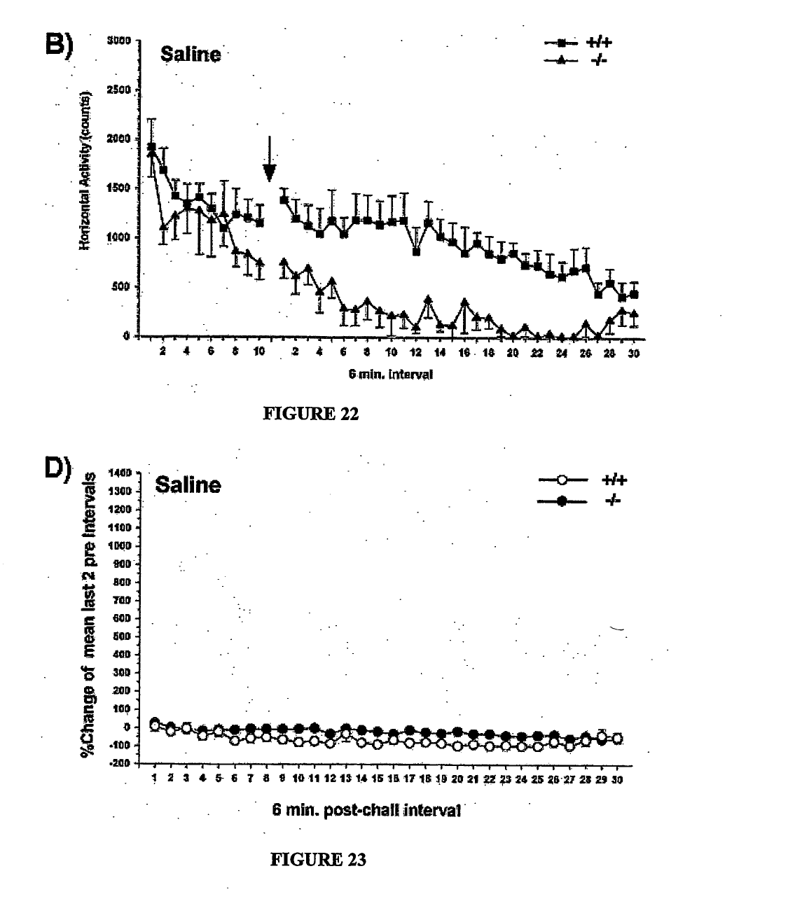

a. Horizontal activity of Npas3+/+(squares) and Npas−/− (triangles) following methamphetamine administration is shown in FIG. 20, and % change in locomotor activity is shown in FIG. 21. Administration of methamphetamine increases locomotor behavior in Npas3+/+ mice, but not Npas3−/− mice (*p<0.05 and **p<0.01). The failure of methamphetamine to increase locomotor activity in Npas3−/− mice is consistent with alterations to dopamine signaling pathways in the mutant Npas3−/− mice. Administration of saline does not increase locomotor activity (FIG. 22) or alter % change in activity (FIG. 23) in mice of either group.

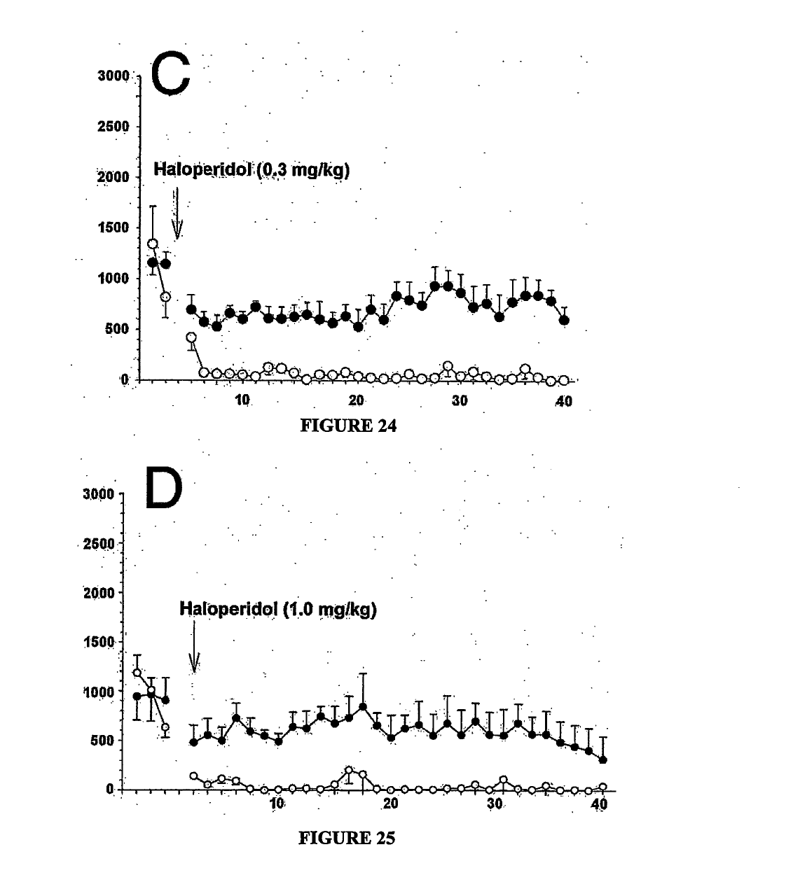

b. Administration of 0.3 mg/kg haloperidol results in substantial impairment in locomotor activity in Npas3+/+ mice (open circles), FIG. 24. In contrast, administration of haloperidol does not reduce the activity of Npas3−/− (solid circles) mice to wild-type levels. However, there is a partial reduction in activity of Npas3−/− mice. Similar results are obtained with a higher dose of 1.0 mg/kg haloperidol, FIG. 25.

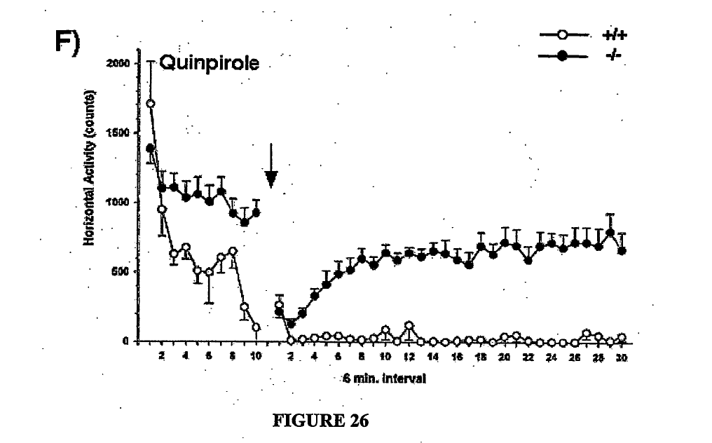

c. Quinpirole inhibits the locomotor activity of Npas3+/+ mice (open circles) throughout the duration of the test period, FIG. 26. Initially, quinpirole inhibits the locomotor activity of Npas3−/− mice (solid circles). However, this inhibition is only transient, as Npas3-−-mice resume activity within 15 minutes.

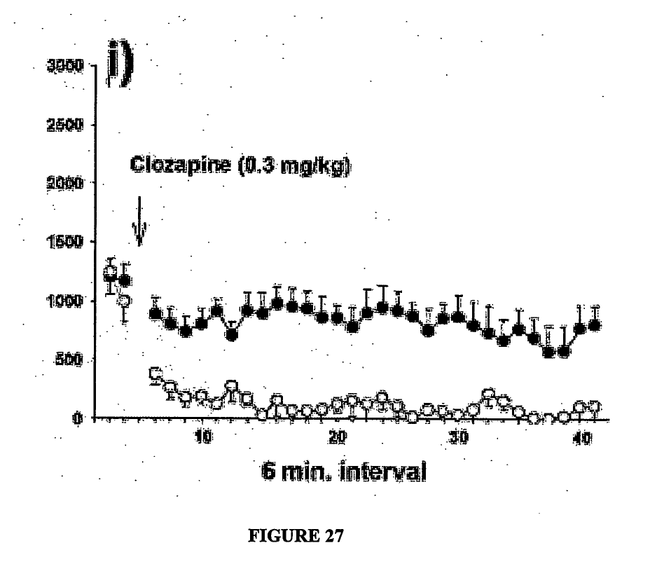

Clozapine significantly reduces the motor activity in Npas3+/+ control mice (open circles), FIG. 27. In contrast, this dose of clozapine is ineffective at attenuating the motor behavior in Npas3−/− mice (solid circles).

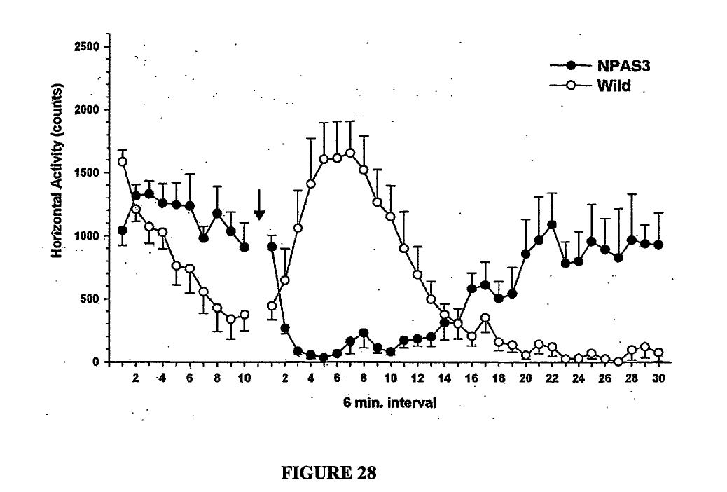

MK-801 increased locomotor activity in Npas3+/+ mice (open circles) but induced hyperstereotype in Npas−/− mice (closed circles), FIG. 28 (p<0.0001). Npas3−/− mice showed a dramatic suppression of locomotor activity to MK-801, presumably because Npas−/− mice had progressed beyond hyperactivity to hyperstereotype. In contrast, this dose of MK-801 led to an increase in locomotor activity of Npas3+/+control mice, but did not increase the levels of stereotype. A lower dose (0.1 mg/kg) of MK-801 potently activated locomotion in Npas3−/− mice, but did not induce hyperactivity in Npas3+/+ control mice, indicating a shift in responsiveness to MK-801 in Npas3−/− mice (not shown).

### MK-801 Binding Assay Protocol:

The exaggerated response of Npas3−/− mice to MK-801 is reminiscent to the exacerbation of symptoms seen in schizophrenic patients exposed reversible NMDA antagonists such as PCP or ketamine. The pronounced catatonia produced by administration of MK-801 is similar to that in mouse models that express reduced levels of the NMDA receptor, that the induction of catatonia in the Npas3−/− mice is due reductions in the levels NMDA receptor. To measure the levels of the NMDA receptor, NMDA receptor density is measured in a MK-801 binding assay. Tissues from hippocampus, cortex, or striatum are dissected from Npas3−/− and Npas3+/+ mice, pooled, and homogenized in a binding buffer of 20 nM HEPES, 1 mM EDTA (pH 7.0) with 100 micromolar glutamate, glycine, and spermidine as described in Nankai et al, 1996, Neurochm. Int 29, 529-542. Binding assays included 80 micrograms of membrane protein and 2 nM [3H]-MK-801 in a volume of 150 microliters. Tubes were incubated at 32 C for 3 hr to reach equilibrium binding. Nonspecific binding was determined by 10 microMolar MK-801. Bound ligand was separated from free by rapid filtration onto Whatman GF/B filters and measured in a scintillation counter. The bound portion of MK-801 is indicative of the level of specific binding to NMDA receptors

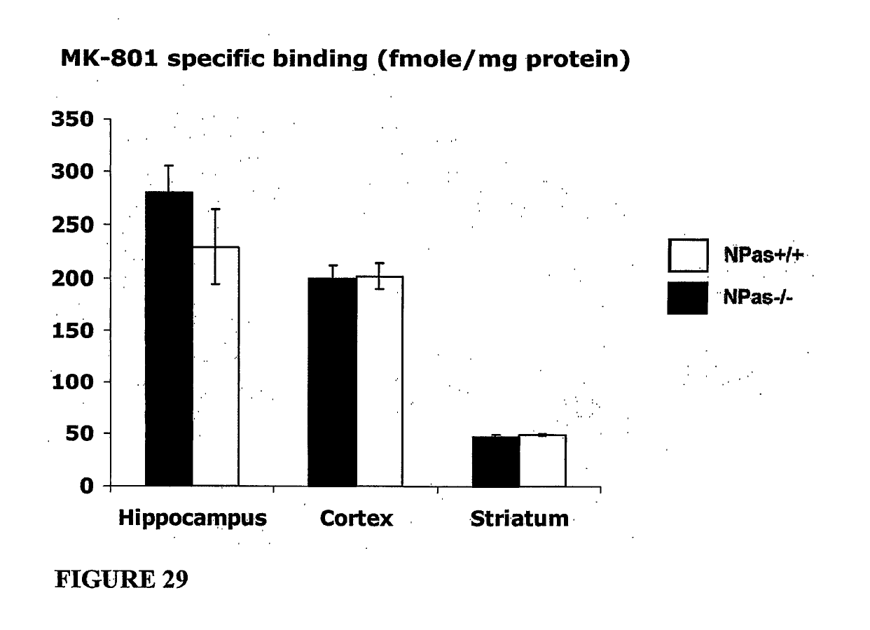

Results of the MK-801 Binding Assay in Npas3−/− mice: This analysis reveals that there is no statistically significant difference in the amount of NMDA receptor in the hippocampus, cortex or striatum between Npas3−/− mice and Npas3+/+ control animals, shown in FIG. 29.

### 4. Biochemical Analysis of the Npas3−/− Mouse:

The inability of haloperidol and clozapine to effectively attenuate the increased locomotor behaviors of Npas−/− mice suggests that dopamine or serotonin signaling pathways are significantly altered. To detect alterations in synthesis of dopamine and serotonin, a detailed neurochemical analysis is performed. The concentration of dopamine, serotonin and their metabolites in several regions of the brain are measured by HPLC. The protocol for Analysis of Neurotransmitters is given below, and results for the baseline status of these neurotransmitters in Npas3−/− and Npas3+/+ mice follow. This Analysis of Neurotransmitters Protocol is also used in practicing the invention as a method for screening a biologically active agent or agents and will be referred to as such in examples of that embodiment. Analysis of Neurotransmitters Protocol.

Brain regions from each mouse are dissected and homogenized individually in 0.1 M HClO₄ containing 100 ng/ml 3,4-dihydroxybenzylamine (DHBA). The brain regions selected for analysis may be any that are of interest due to association with schizophrenia or related neurological disorders. Following centrifugation at 10,000×g for 10 min, the supernatants are filtered through 0.22 mm filters and analyzed by HPLC-EC for levels of the neurotransmitter dopamine (DA) and its metabolite dihydroxyphenylacetic acid (DOPAC), and the neurotransmitter serotonin (5-HT, 5-Hydroxy-tryptamine) and its metabolite 5-hydroxyindole acetic acid (5-HIAA) See Wang et al. (1997) Neuron 19(6): 1285-96 for more detailed mythology.

Results of Analysis of Neurotransmitters in Npas3−/− mice: Results are shown in Tables 1 and 2. Though some regions of the brain, including hippocampus and prefrontal cortex are found to have normal levels of dopamine, serotonin and their metabolites, there are 58% and 65% reductions of dopamine and DOPAC, respectively, in the striatum of Npas3−/− mice compared to Npas3+/+control mice. In addition, there are 27% and 31% reductions in the amount of dopamine and DOPAC, respectively, in the anterior cingulate cortex of Npas3−/−mice compared to Npas3+/+ control mice. In the hypothalamus, 5-HT is increased by approximately 28% in Npas3−/− mice compared to Npas3+/+ control mice. Asterisks indicate significant differences between Npas3+/+ and Npas3−/− mice: * p<0.08, **p<0.05 and *** p<0.01.

TABLE 1
5-HT and 5-HIAA concentrations in males, Mean ± SEM (N) expressed
as ng/mg tissue.

| Region | Monoamine | WT | Npas3^{-/-} |
|---|---|---|---|
| Striatum | 5-HT | 0.35 ± 0.04 (8) | 0.39 ± 0.05 (10) |
| | 5-HIAA | 0.22 ± 0.02 (8) | 0.21 ± 0.03 (10) |
| | Ratio | 0.62 ± 0.02 (8) | 0.56 ± 0.04 (10) |
| Anterior Cingulate | 5-HT | 0.18 ± 0.02 (9) | 0.17 ± 0.02 (9) |
| | 5-HIAA | 0.08 ± 0.01 (9) | 0.08 ± 0.01 (8) |
| | Ratio | 0.46 ± 0.04 (9) | 0.43 ± 0.05 (8) |
| Prefrontal cortex | 5-HT | 0.68 ± 0.05 (9) | 0.62 ± 0.03 (10) |
| | 5-HIAA | 0.17 ± 0.01 (9) | 0.20 ± 0.02 (10) |
| | Ratio | 0.27 ± 0.03 (9) | 0.33 ± 0.05 (10) |
| Hypothalamus | 5-HT | 1.15 ± 0.04 (9) | 1.61 ± 0.06 (10)*** |
| | 5-HIAA | 0.48 ± 0.02 (9) | 0.59 ± 0.05 (10)* |
| | Ratio | 0.42 ± 0.02 (9) | 0.37 ± 0.02 (10)+ |
| Hippocampus | 5-HT | 0.60 ± 0.04 (9) | 0.58 ± 0.04 (10) |
| | 5-HIAA | 0.42 ± 0.03 (9) | 0.34 ± 0.04 (10)+ |
| | Ratio | 0.75 ± 0.10 (9) | 0.57 ± 0.02 (10)+ |

***P < 0.001
*P < 0.05,
+P < 0.10.

TABLE 2
Dopamine (DA) and DOPAC concentrations in male mice, Mean ± SEM
(N) expressed as ng/mg tissue.

| Region | Monoamine | WT | NPas3−/− |
|---|---|---|---|
| Striatum | DA | 6.30 ± 0.95 (8) | 4.64 ± 0.56 (10) |
| | DOPAC | 0.63 ± 0.06 (8) | 0.58 ± 0.08 (10) |
| | Ratio | 0.12 ± 0.02 | 0.17 ± 0.06 (10) |
| Anterior Cingulate | DA | 0.03 ± 0.005 (9) | 0.02 ± 0.003 (9) |
| | DOPAC | 0.02 ± 0.001 (9) | 0.01 ± 0.001 (9) |
| | Ratio | 0.69 ± 0.12 | 0.66 ± 0.07 (9) |
| Prefrontal Cortex | DA | 0.14 ± 0.04 (9) | 0.22 ± 0.07 (9) |
| | DOPAC | 0.04 ± 0.005 (9) | 0.06 ± 0.01 (9)* |
| | Ratio | 0.44 ± 0.09 (9) | 0.41 ± 0.0 (9) |
| Hypothalamus | DA | 0.34 ± 0.03 (9) | 0.45 ± 0.04 (10)* |
| | DOPAC | 0.13 ± 0.01 (9) | 0.16 ± 0.01 (10)+ |
| | Ratio | 0.40 ± 0.03 (9) | 0.37 ± 0.02 (10) |

*P < 0.05,
+P < 0.10

In addition, brain regions, cell, or groups of cells, and non-neuronal cell or groups of cells from any tissue of the Npas3−/− mice can be used to measure or detect changes in synthesis, phosphorylation, dephosphorylation, protein processing, catalytic activity, or other characteristics of protein or gene function associated with schizophrenia or related neurological disorders. These can include but are not limited to GABA, dopamine, and serotonin receptors, downstream signal transduction molecules, cyclic-AMP (cAMP), protein kinase C (PKC), and transcriptional regulation of target genes.

### Prepulse Inhibition of Acoustic Startle Protocol:

Prepulse inhibition of acoustic startle (PPI) is a reduction in a startle reflex induced by a prestimulus due to impaired sensorimotor gating. Deficit in PPI is associated with schizophrenia in humans and is a conserved reflex found in animals. PPI is been routinely used to validate the ability of animal models to represent the human condition. In general, a high decibel (dB) auditory stimulus (sudden loud or intense tone or sound) produces a startle response in animals with normal hearing. However, if the high dB1 stimulus is preceded by a stimulus of less intensity (“prepulse” stimulus sound) the startle response to the subsequent high dB stimulus will be lessened.

The test comprises a measurement of the animal's response to a sound of 115 dB to establish the baseline. The test delivers a prepulse sound of 74, 76, and 78 dB, followed by a sound of 115 dB, and is a measure of the animal's response to the 115 dB sound under the prepulse conditions. The PPI is a mathematical measure of inhibition of the startle response to a stimulus of 115 dB preceded by a prepulse stimulus, and is expressed as a percent of the baseline startle response.

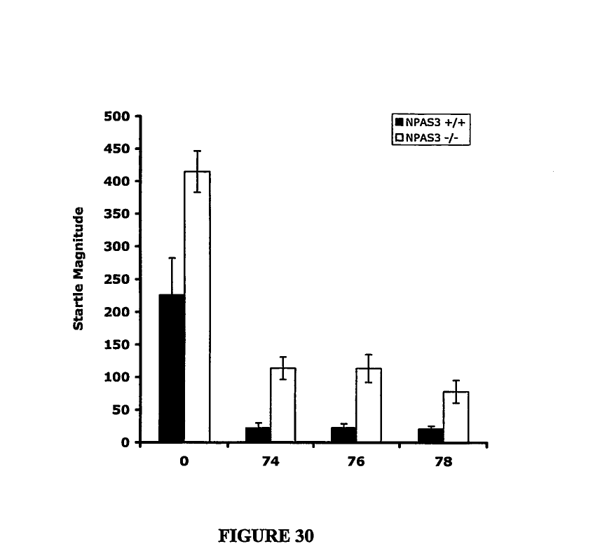

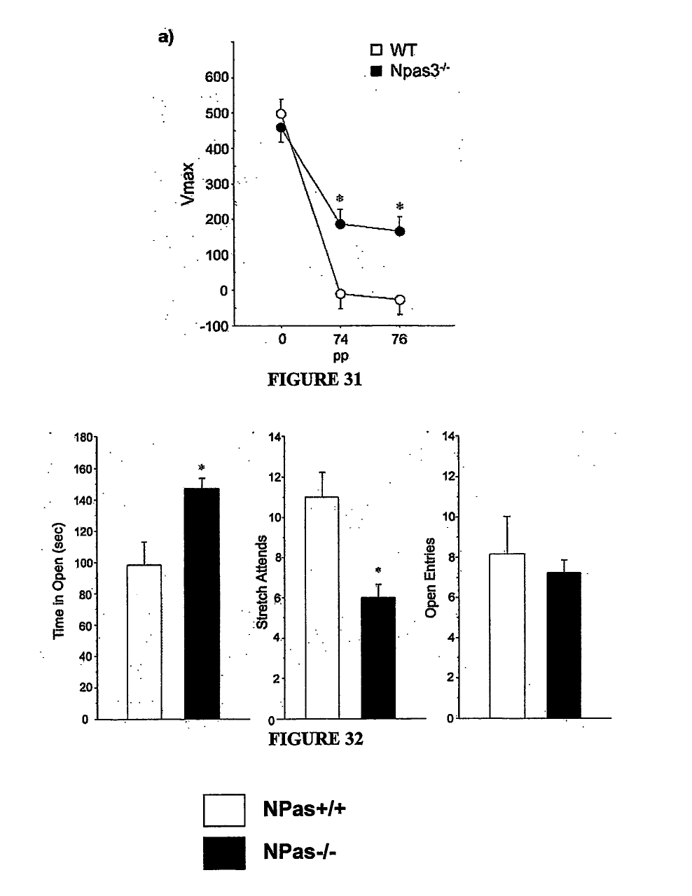

Results of PPI test in Npas3−/− mice: Npas3−/− mice (white bars) have a markedly elevated baseline startle response compared to Npas3+/+ controls (black bars), as shown by the 0 dB Prepulse in FIG. 30. Startle response following prepulses of 74, 76, and 78 dB are also shown in FIG. 30. During PPI, Npas3−/− mice (solid circles) show a 69% inhibition of acoustic startle response when prepulse intensities of 74 dB and 76 dB are applied, shown in FIG. 31. In comparison, the same prepulse levels inhibit startle reflex by 90% in Npas3+/+controls (open circles). There is no statistical difference between groups with a prepulse of 78 dB (not shown). This test demonstrates that Npas3−/− mice have impairments in sensorimotor gating, similar to those found in patients with schizophrenia.

### 5. Assessment of Anxiety

Anxiety and fear are natural adaptive consequences of stress that help to prepare for coping with the stressor. However, anxiety disorders are chronic, persistent, and can grow progressively worse if not treated. Fear and anxiety are not uniquely human emotions. Rodents show a similar pattern of behaviors in fear provoking situations, including increased heart rate and blood pressure, decreased eating, defecation, behavioral immobility and increased startle. The similarity of these signs of fear and anxiety as well as the similarity of the situations that elicit them provides a means to examine the neurobiological basis of fear and anxiety in non-human models.

Zero Maze Test: The zero maze comprises a doughnut-shaped ring with interior and exterior walls that cover 90 degrees of the rings circumference on opposite sides. In this task a mouse is placed in a closed portion of the maze. From here the mouse can walk along the ring in any direction. The amount of time spent in the open portion of the ring and the number of times the mice enter the open portion of the ring are recorded. “Anxious” mice will spend little time in the open portion of the ring and make very few entries into the open portions of the ring. Anxiolytic drugs known to reduce the subjective feelings of anxiety in humans increase the time spent in the open portions of the ring.

The elevated zero maze consists of a ring 50 cm in diameter. The width (surface area) of the ring is 8 cm. The height of the walls on the closed arms is approximately 18 cm. The maze is placed in a dimly lit room and was elevated 1 m above the floor. A mouse is placed in a closed portion of the ring and continuously observed for a period of 5 minutes. During the 5-min observation period the cumulative amount of time the mouse spent in the open portions of the ring and the number of entries into the open area is recorded. An open ring entry is defined as all four paws in the open portion of the ring. “Stretch-attend” movement is a stereotypical movement that a mouse makes when investigating an unfamiliar area, stretching its snout forward, and pausing to observe before moving forward. Cumulative time in the open portion of the ring is recorded only when all four paws are in the open portion of the ring. At the end of the 5-min observation period the mouse is returned to its home cage. The amount of time (in seconds) spent in the open portion of the ring and the number of open ring entries are recorded for each mouse. The cumulative amount of time spent in the open portion of the ring is converted to the percent time in open portion by dividing the cumulative time by 300 seconds (i.e., 5 minutes) and multiplying by 100.

Results of the Zero Maze Test in NPas3^{-/-} mice: APas3^{-/-} mice (black bars) show a 50%increase in the time in the open quadrants and 54% reduction in stretch-attend movements at the boundaries, compared to NPas3^{+/+} mice (white bars), as shown in FIG. 32. This combination is consistent with a diazepam-like anxiolytic effect. In effect, NPas3^{-/-} mice enter open areas with less hesitation and remain in the open longer. NPas3^{-/-} mice are hyperactive on a test of locomotor activity, however, in the zero-maze the number of open entries (an index of activity) is not increased. In fact, APas3^{-/-} mice showed a slight decrease (p <0.10) compared to NPas3^{+/+}. NPas3^{-/-} mice enter the open areas less frequently but when they do enter they remain there longer on each occasion. This suggests that the NPas3^{-/-} mice are not globally hyperactive, but rather exhibit context-specific changes in activity, such as those associated with schizophrenic behavior.

### 6. Method for Analysis of Spatial and Recognition Learning and Memory

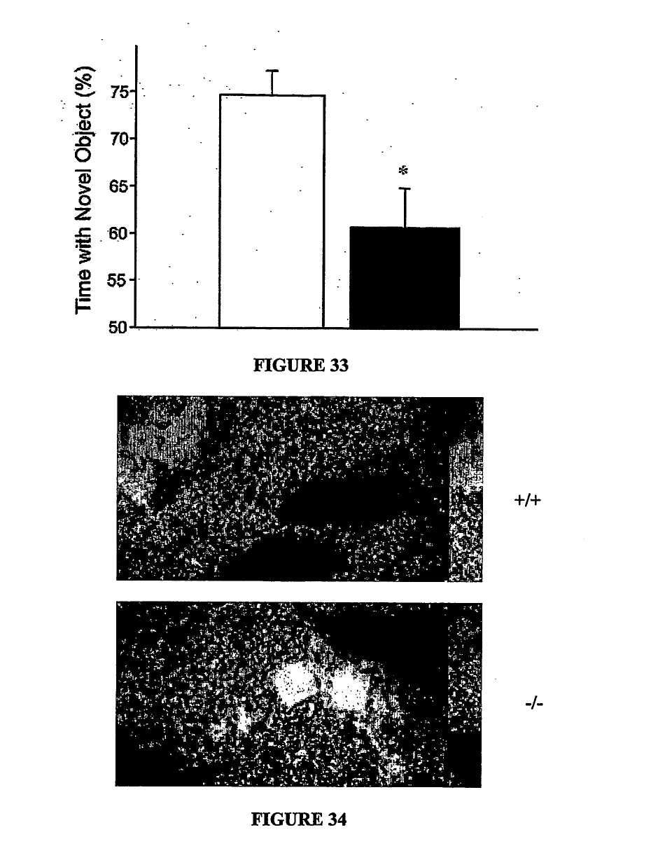

Novel Object Recognition Test: The hippocampus is a region important for both spatial and recognition learning and memory. In order to test if the reductions in hippocampal volume observed in the NPas3^{-/-} mice affected recognition learning the animals were tested in a novel object-recognition task. This task measures visual recognition memory and in evolutionarily conserved in species including humans and rodents. Results of Novel Object Recognition Test: As shown in FIG. 33, the Npas3−/− mice (black bars) investigate the novel object significantly less (61% of the time) than Npas3+/+ mice (white bars, 75%). This result indicates significant impairment in recognition learning.

### 7. Method for Evaluation of Nesting Behavior

Nesting Behavior Test: When nest materials are place in a cage, mice will shred the material and arrange it into a stereotypical nest shape. Failure to assemble the materials into a nest is indicative of neural deficits associated with neglect of pups and failure to nurture and care for pups.

Results of Evaluation of Nesting Behavior: As shown in FIG. 34, when nesting material was placed in cages, Npas3−/− mice (lower panel) failed to build a nest, compared to Npas3+/+ mice (upper panel). Inability to care for or nurture offspring is also a hallmark of schizophrenia.

### 8. Method for Testing the Efficacy of an Agent in the Treatment of Schizophrenia:

An Npas3-deficient mouse is generated, for example, as described in Section 2 above. The resultant Npas3−/− mice can be assigned to 2 groups for treatment with either a test agent or a control agent, such as saline or other vehicle for the test agent. The number of mice in each group needs to be sufficient for determining statistical significance of the results of each test. This number can typically be 8 to 12 mice. It is preferred that test and control groups of Npas3+/+ mice are treated along with the Npas3−/− mice to provide a “normal” or “wild-type” response or performance for comparison. Prior to administration of the agent or control substance, mice are tested to determine baseline response or performance in behavioral phenotype using a test or combination of behavioral tests, selected, for example, from the group comprising the Tail Suspension Test, Footprint Test, Beam-walking Test, Locomotor Activity Test, PPI, and Zero Maze Test. Mice can also be killed at appropriate time points for Analysis of Neurotransmitters or other biochemical assays. Following administration of the test agent or control substance, mice are tested for changes in response or performance using the same test or tests used to determine the baseline behavioral phenotype. Agents that improve the response or performance on the behavioral tests or biochemical assays are interpreted to be potentially therapeutic for schizophrenia or related neurological disorders.

### 9. Method for Screening Agents for the Treatment Schizophrenia:

An Npas3-deficient mouse is generated, for example, as described in Section 2 above. The mouse is then used as a source of a cell or cells with alterations in one or both Npas3alleles. These could include any brain region associated in schizophrenia or any related disorder. However, any cell from any other tissue can also be of interest, and thus can be derived from any region of the mouse.

In one embodiment, the substantia nigra is micro-dissected from the brains of 20 Npas3−/− mice on fetal day 19. Tissues are treated with a cocktail of proteolytic enzymes, and cells are gently dispersed in media and allowed to incubate at 37° C. for 4 hours. Cells are pooled, counted, and distributed evenly in 6-well tissue culture plates. A biologically active agent added to the media in three of the wells. Cells are cultured at 37° C. overnight Media and cells are harvested in 0.1 M HClO₄ containing 100 ng/ml 3,4-dihydroxybenzylamine (DHBA), and tested for changes in dopamine and DOPAC levels as described in the Analysis of Neurotransmitters Protocol. An agent is identified as a potential therapeutic agent for schizophrenia or related neurological disorders if it increases dopamine and/or DOPAC levels compared to levels measured in untreated cells, or if it results in more normal synthesis of another neurotransmitter, or if it results in a more normal downstream signal transduction process for another neurotransmitter.

In another embodiment, the anterior cingulated cortex is micro-dissected from the brains of 20 Npas3−/− mice on fetal day 19. Tissues are treated with a cocktail of proteolytic enzymes, and cells are gently dispersed in media and allowed to incubate at 37 C for 4 hours. Cells are pooled, counted, and distributed evenly in 6-well tissue culture plates. A biologically active agent added to the media in 3 of the wells. Cells are cultured at 37° C. for 0.5, 2, 4, and 24 hours. Cells and media are harvested and proteins and RNA are extracted. Protein fractions are used to determine changes in GABA, dopamine, and serotonin receptor levels, cAMP levels, or changes in phosphorylation of downstream signaling molecules in the receptor pathways. An agent is identified as a potential therapeutic agent for schizophrenia or related neurological disorders if it modulates synthesis or phosphorylation of any of these receptor pathway molecules compared to that in untreated cells, or if it results in more normal synthesis of another neurotransmitter, or if it results in a more normal downstream signal transduction process for another neurotransmitter. RNA isolates are used to determine changes in transcription of target gene. Increased or decreased levels of MRNA transcripts of individual target genes are used to identify agents that restore a profile of gene transcription associated with normal or wild type patterns of gene expression.

In another embodiment, stem cells are isolated from the blood of an Npas3−/− mouse and treated with growth factors to induce a neural phenotype. These cells are then treated and assayed for biochemical changes.

In yet another embodiment, cells are derived from peripheral tissues, such as muscle, isolated, treated and assayed for changes in phophorylation and synthesis of cellular proteins or gene expression contributing the phenotype exhibited by Npas3−/− mice or associated with schizophrenia or related disorders.

### 10. Method for High-Throughput Screening of Agents for Treatment of Schizophrenia:

An Npas3-deficient mouse is generated, for example, as described in Section 2 above. Immortalized cell lines are then made from the transgenic mouse. Any so-called “transformed” immortalized cell can be used to develop such a cell line. This can be achieved using a wide variety of techniques well known to those skilled in the art, including transduction with an oncogene via plasmid or viral vector, treatment with teratogenic or mutagenic agents, fusion with another immortalized cell to form a hybridoma, or irradiation. In one embodiment, the Npas3-deficient mouse is crossbred to a transgenic mouse with tissue-specific expression of SV40 Large T Antigen. SV40 Large T Antigen is well known to those skilled in the art of cell culture as an agent that will cause tumor formation in mice. Npas3−/− cells expressing SV40 Large T Antigen can be isolated from tumors or tissues without tumors to develop clonal populations of single transformed cells, thus developing an immortalized cell line from the tissue source of interest. These could include any brain region associated with schizophrenia or any related disorder. However, any cell from any other peripheral tissue may also be of interest, and thus may be derived from any region of the mouse.

In one embodiment, tumors forming in a discrete brain region, for example the anterior cingulate cortex, are micro-dissected from the brains of adult Npas3−/− mice. Tumors are enzymatically digested, and cells are gently dispersed in media and allowed to incubate at 37 C for 4 hours. Cells are then diluted and distributed in 96-well tissue culture plates so that each well contains, on average, only one cell. Wells are monitored and subdivided periodically to develop clonal populations of single cells. Candidate clones are analyzed for expression of Npas3 and marker genes specific for anterior cingulate cortex neurons. The resultant cell line is amplified, and frozen stocks of the cells are stored in liquid nitrogen. When a sufficient quantity of cells is obtained from a single cell line verified to be Npas3-deficient and of anterior cingulated cortex lineage, the cells are plated out into 96-well plates. Triplicate wells are treated with small molecules from a library of such molecules or with control substances such as media or phosphate-buffered saline. Cells are harvested at various time points following treatment, and phosphorylation state of signaling transduction molecules and levels of cAMP are measured. Any agents that induce changes in phosphorylation levels or cAMP levels are identified as potential therapeutic agents.

In another embodiment, cell lines are derived in a similar manner from the substantia nigra. Plates of cells are treated with a variety of agents and incubated overnight at 37° C. Media and cells are harvested in 0.1 M HCl0₄ containing 100 ng/ml 3,4-dihydroxybenzylamine (DHBA), and tested for changes in dopamine and DOPAC levels as described in the Analysis of Neurotransmitters Protocol. Any molecules that increase dopamine and/or DOPAC levels compared to that of untreated or control cells are identified as potential therapeutic agents for schizophrenia or related neurological disorders.

In other embodiments, cells from any other region of the brain associated with schizophrenia or any related neurological disorder can be used as a source of cells from which a cell line can be derived. Similarly, the cells can be treated and used to test for normalization of the synthesis of other neurotransmitters, and for the normalization of neurotransmitter downstream signal transduction pathways that play a role in schizophrenia or related disorders. Cells may also be stably transfected with reporter constructs that will allow the user to more conveniently detect biochemical changes in concentration of a molecule such as cAMP, PKC, or other proteins or protein modifications, or in expression of target genes. Suitable reporters can provide a wide variety of read-out signals, including, but not limited to luciferase, -galactosidase, and colorimetric changes that can be detected spectrophotometrically. Embodiments such as these are the basis for high-throughput assays that are particularly useful for rapid screening of libraries comprising hundreds or thousands of molecules or compounds design to act upon a wide variety of targets.

1. A transgenic mouse having a genome that comprises a mutation of an endogenous Npas3 gene, wherein the Npas3 mutation causes a disruption that inactivates the gene, and a homozygous transgenic Npas3 mutant mouse does not produce a fully functional NPAS3 protein, wherein the transgenic mouse exhibits a phenotype that models schizophrenia.

2. The transgenic mouse of claim 1 wherein the disruption of the endogenous Npas3 gene comprises a deletion of at least one exon of the Npas3 gene, replaced with heterologous DNA sequence.

3. The transgenic mouse of claim 1 wherein the disruption comprises a conditional disruption that is regulated by an inducible factor.

4. The transgenic mouse of claim 1 wherein the exhibited phenotype is selected from the group consisting of dyskinesia of hindlimb and foot-clasping posture, parkinsonian gait of stride length and footprint pattern, altered neurotransmitter signaling selected from the group of neurotransmitter consisting of dopamine, seratonin, GABA, and glutamate, altered neurotransmitter signaling pathway selected from the group consisting of dopamine and seratonin; altered responses to glutameteric signaling pathways such that administration of a glutamate analog induces hyperstereotypic behavior, and combinations thereof.

5. At least one cell derived from the transgenic mouse of claim 1.

6. A method for determining the effectiveness of a biologically active agent in a transgenic mouse, comprising the steps of:
a. disrupting the at least one allele of an endogenous Npas3 gene in the transgenic mouse wherein the disruption inactivates the gene,
b. administering to the mouse the biologically active agent, and
c. assessing for a change in a phenotype of the mouse, wherein the phenotype is a phenotype selected from the group consisting of dyskinesia of hindlimb and foot-clasping posture, parkinsonian stride length and pattern, impaired balance and motor coordination on a narrow beam, impaired locomoter activity, hypersterotypic behavior, impaired prepulse inhibition, impaired zero maze behavior, altered gene expression, altered protein synthesis, altered receptor activity, elevation of cAMP level, altered protein dephosphorylation and altered protein phosphorylation, and combinations thereof.

6-8. (canceled)

10. A method for determining the effectiveness of a biologically active agent in a cell line derived from a transgenic mouse, comprising the steps of:
a. disrupting at least one allele of an endogenous Npas3 gene in the transgenic mouse wherein the disruption inactivates the gene,
b. isolating at least one cell from the transgenic mouse,
c. deriving an immortalized cell line from the isolated cell,
d. amplifying cells of the cell line,
e. administering at least one biologically active agent to the cells of the cell line, and
f. detecting a biochemical change in the cells of the cell line.

11. The method according to claim 10 wherein the isolated cell is a neuron isolated from a brain region selected from the group consisting substantia nigra, striatum, hippocampus, anterior cingulate cortex, and prefrontal cortex.

12. The method according to claim 11 wherein the biochemical change is selected from the group consisting of changes in synthesis of dopamine or dopamine metabolites, gene expression, protein synthesis, receptor activity, elevation of cAMP level, protein dephosphorylation and protein phosphorylation, and combinations thereof.

13. The method according to claim 11 wherein the amplified cells of the cell line are placed in at least one multi-well culture plate for high-throughput screening of a number of biologically active agents.

14. The transgenic mouse of claim 1, wherein the transgenic mouse exhibits a phenotype that models schizophrenia with a mutation only of the endogenous Npas3 gene.

15. The transgenic mouse of claim 2, wherein the heterologous DNA sequence comprises a gene expression cassette that confers antibiotic resistance to a host organism.

16. The transgenic mouse of claim 3 wherein the inducible factor selected from the group consisting of Cre-recombinase in a Cre-lox system, Flpase in a FRT-Flpase system, and combinations thereof.

17. The transgenic mouse of claim 5 wherein the cell is a neuron isolated from a brain region selected from the group consisting of substantia nigra, striatum, hippocampus, anterior cingulate cortex, and prefrontal cortex.


SEQUENCE LISTING (1 sequence)

SEQ ID NO: 1 - DNA, 20000 bp, Mus musculus
  misc_feature (1175)..(1194): n is a, c, g or t
agtgcttaaagacatggagggacccatggctccagctgcatatgtagcagaggatggccttatctagcatcaatgggacgggagggcccttggtcctatggaggcttagtgccccagcgtagggaaaaatgggggtgggtgggtgggttggggagcatcctcatagaagcaggggaaagggggataggataggagtttgcaaagggggaaagcaggaaggggaatagcacttgatatataaataaataaaataaccagtgcaaatagatagataaaaaaatcatatgagcaaaactaaaagggaaaaaaagaaggaatgcagacagttaaattatacagatgtcctccttaacattagtaaggcatggcatgtgtgtggtggtcacatgatcataactttattttcacacttaagaatcttcagacttgtcaaagcatgcttgctgttttccaagtgagccgcagcagacttgaaaggtttaaacagttctttcactaagttgtgctttcaagtgtttgaaatcacacttgaagaaaattgaaagaaaaagtaaaatttcccttaattaaaaaaaaaaagtcattttgaatttgaattccaaagcaatagagtttgcgatttgaagcaattaatagcatcaccagaagcctgaaaatggagattttgttgaatatttcattaaagaattaagctttttgtaacaattattagggaatctctacaggtaattgtaaattaaacccagctttatcagtgttgtagaagttcaaaagacaaaacagaccatgacatcttatgcctgcagaggcatggtgatgctctggtgacaatgacaggcttgtgccccttgccaagggtcaccttgtaaaagccactcccgctctctactttgaatcctgatggtaggaatcagctatcctgatttatctaggatttttccacgtggagcacaagacatccaacatccaaagaaggacctaccagagccaaagttactccctggttttagggatggaagaggacacaaactcctaccccctatcttacctgcaacgaaagagacatgattcatgatcaaaagcagctcggattcagtagtggatgggtaggtgtgagtcacctaccatccttagtaactgatactggctaactgcttccctgctgataactgtannnnnnnnnnnnnnnnnnnnagctagaaatgccagcacccccatttgtggggccaggaaagtctctttagccaaaggtggccaccttagcttttctctttgcctttcagcagcgatccaaagcacctttcaaatatttggcagcagcatcctctcaatgtgtgctcacacctcctgttacaattaatggaagtgacatatgtgtgctcaccaggaggagaaaggctttatcccatgttggacacagacccgtgatcctgaatctctttctaaacttcagacctgaggagaacagtactgggaattgctgctttctcactacattagcgacttcaatttccttttaattggagcctgcaatccagatccttggaacttcagggggtgaacaatatcaattaagatctagatgctttctaggaagtgctagttcagagagtgcctttctttccttctgtcttctttctttctttctttctttctttctttctttctttctttctttcttccttccttccttccttccttcctgtctttctgtctttttttttttttttgcttctttaaacacagttgtggccattcacaaaacacatgcagtggcctaatgagttcatatttacttctgaggtttcacatttgcaaaccacactgctgggaaaataagtgaagtgtttctcaaccaccactgcgagctaacaggcttaatgtacagggcagataaatacttgaattgtcaaaatcccccaacagtgcacagagccttcagatatagcatcttccccttgttcaggacattcatttctcccaagaattctttttaatgtaggacttcaatttatgtttaagaactagtaaactatagtggtctgacagccattcacacttaattctgtgcattaaaccaacagcattcttttctacaaacatccattgttaggacaatgctcttagacagaatttatgagaaatggaattatgttaccaaggagatagcatttactataatgaagagtagatcctggaaaatggccccttttacagcagctcagagcagagccacaaaagcaattgattggtcttggcatttagtcttatatcagctgtgtcatccatagcttcaggagtgagaacactaatctaaagtgacatttctgttctggcgggcttttctgccctgatgaaagcaattacttcatccctaaatgataatttgcaggagtttcaaccactttaattgataatttttttgttgttgttctgagttcaattgtgaagtgattagctgattttgtaaataggaatttaaggtgtgaaatatacaagctgccccaaggtaaagtgctatttattgggtaaagagaaattacttcataaagctgcaggttttccccccattattgtgaaactggcagttgtgtatgttgaagttgtaggataaaaaaaaccaaagaacaaaacactttgattcacgagactatgggcacacagctaagcgcgtgatgaatagcttggtcttgttgtggttcattcaagggttatttgaaccaaggtgaacgctagttgaaaggggctgggcagactgatcagctttccttattactgtactaatgactcattctggagtgagagcctcttatttaaagtggtaagaatgaaaaacgctcattggctaaaaccaattaatttaattattaagttcaaacgtacacaagacattttctgaagatgatgggaggcattccgatgaatggctttaattactcgtctgatctactttgtgcctcaagtcaaaagggtcatgatgatcccatcggaaaatgccttagcttaaaaaccagaagcatgtctcttggttttggtactttacgaaacgttagcaatcagatgttttttgacgctgcttagaagcaatcagtttcaatattttcttacatgtggagacacagtggtcatgtcacatgttaaatttgaaaaataaagcaggagtgaaggtcagactgatttagcacatcaaacagtctttgtcagccaagcttaatatatcctgagtttgtggaacaaatgaaattttgctgctgttacactttgcttccaaacaaccattacaactggtttcatatattgtgtgtgcatcagaccatcaagctgtgtgtctctactgacgtgttgctgcggcaatgatctacagtggcaatctgccatttatataagggcaaaatgtgcatctatatttagttgtgaaattggcctattatactcctgatgtttacagaggctaacataaattaatgatgacagctatgagccgtgcattagttaagcagcagagttcttaccttgaacttgaaggttccagttttaacatctagtatagactaccctgcttgcttcacgtgctaaatgaagatttctgaaatgattgccgtctcttgagctctgaaatacttccattggtggactttgttgttcacactctcaccaggtaatagtttcctatccatttctcaactgtactaccttgcatttacatctattctaaaattacacccaaaagagcttattctgcagcaaacggatttgcttgactttgaattctatgtgatgtcagtgtggggagggctgttttactaattgtaatgaaggaaggaccagtgtgcttatggggcctctattgtatggttttcttgaaaggccctctcattcagctcatgtcagggcaagtgtaggtactggagtagctgtggactttaccattaactatggccagagatctgacaagtgtgcaggtgttcaaaggttcatgagttaaaatgctgaagcctttgatgaatttttgatggctgcatttttaatacaggtctaccttcttcctttgaggctgcatggcagattaattgataaataggagtctgtagttctgggtcctcctaagcaaaacaagtaggaaaatccataaaatcttgcctgcctggctccttagaagtaccttaaaaaggcaaaatttaaagccctttcctctgctcctttctgcctctatagggggatagccaaaccctgccatgctctttcagaaagactacatattagattgtaaaactttccacaaagtattcaaactgaggcttagtggcgaggtttccgtggagaggggtaacattggactgataacatcaatagcaaaggaaatgaatcaaaacatttacattacaaaatacattttatcttgattattattaatattgttgttatttaatttttttacagtcccagtggttatcctcacccagtctccccttctaccccaccccatctctaagaggatacccccacttcccactcccatcccaccaggtctccccactccctggggcctctagtctctcgagggttaggtgcatcttctctcactgagtcaagaccaggaagtcttctgctgtgtatgtgtcagggacctcatatcagctagtctatgctgcctggttgctggctcagtgtctgagaggtctcaggagtcaggattagttgcgactgttggtcttcctatagggtcgccctcctcctcagcttcttccagcttttccctaattcaaccacaggggttcccagcttctgtccattggttaggtgtaagtatctgcatctgagtctttcagctgcttgttgggcctcttggagggcagtcatgctagtctcatgtttgtaagcaccccatagcatcagtaacaatgtcaggccttgaagtctccccttgagatggatcccaagttgggccaatcaccgaacctccttttcctcagtcccttctccatttttgtccctgcagttcttttagataggaacaattctgggtcagagtttttgactgtggaaaggaaactccatcccttcgcttgatgctctgtctttctactggagatggactttacaagttcactctccctagtgtttggcctttcacctaaggtccctctctgagtcctgcgcatgcctcacctcccaggtctctggtacattctagatggtccattcttccattctgcttccattctttcttctgtccctcaggacttcactcctgtttctcctgccccgaataccttcttatgttccccttcccccctccctgtctcctctcccaaccacgcccatcctggcttgtaagagcatgtctctgaacagatcactgcagaggaagatagtgttggtttattgtgtttccctgttgctcttctgtgattgttgcttatactttgttgttgtccttgttgttggcctttacagttgttttagttcagtaataattttaaccattgtaatgacttcccaaaccaaaaggaatcaattttgcatgtctagtgttgatcatactcaaaagatattgcttccttataatttaatgagtaaccagttcaaagtttgtaaccttcccctgtaatggataagcccaataagtgattgatactcatagctagttccatgtgtcaggaaactgacaatgccctttctttaactgtgatttggttcttctgtttcctcagtttctgtattaaatacagtctctcagtgtgacatgactcttggtgaggagcttgcaggacagagtcattcctggttgtatgagctctccagggtcctggatttgacctcactagaacattcagattagcaagctttggaagatattttgaaacagtcttacttctaagagagcctatgtgttctttataacttaaaattaccaaaatgactatcaccatttaaggtttcctgcacatacttttcaatcttcctgagtaatctatgtctccattataaatgagcactgtggacattcaaagaatcaaactagccatgtgaaattcaattagtctaacatcacatgacttatacggtaactataaacatttcattaaagcattatttctaaaaaataataatacttagtggcatcaccccattctacagattagaaaacccaaggcattaaaattatctttacatagttaataagtaccagagcaaggattaacaccaaatattttgactgaacatttcttgttcttctaataagaactggaaatgtttgcttagtgtttccctcagttggggtgattggaaccatttgaaaatttgacaattacttactgatctcttgaaaaattaacacagaaacacaaccttggttatgatttttggtaaagaaaaaatgtatatgtatatagaaaaatgatgtcttttgaacttatttgttgatcttgttatttgcttgtgatgatcttctttgcagcacaggatggatcagtcaccagatagagttgatactgtcacagtctttgctgatgattaatgggctccctggttcttttatccagaatccaaactgagctcagtagagttctgggatggctatatccagaaagccaataattatggactggaagttttttcatgtttttgatgggagagataggcactgtggtattttcctcctatacaaccaatatatgctgtttctgcattttagaaatgcagcccaaatgtcttgtctagaacaaaatatacagaaacaaagtttattttaggttttaacatggtccttaattcaatttgagaactgattacagctaggctctacttcataaagctgagcagaggattaatccgcaaagctgtgttattgtcttaagatctttagtttcatctattataccagagtctcaacagccattataatcctttgactgtattttattgctgttgccctcaaaaccaattattacttattatagacacagtgctgccattctgaaactcttctgaactatggtgtatgcataatgatgtgctgtgggagtcatggaaaccctatattattaagataatggccaagaacatgacttcagtagaggggtctattgtgtgttagagttgagggagctagtgtgagaaggtggcaaagagaacagggaggtagtgtaggagcttatgaaggtgtgtgtgtgtgtgtgtgtgtgtgtgtgtgtgtgtgtgtgtgtgtacacgcgcttgttaataacattggtcagtgctggcatggaaaagtaagtgaagtatatttagacaaagtgttcatacatgtcagtatctatctattctccttccctgaggctcaaagggttgtgtcccaatcaacccattggaagctactagttggtcttctaccatcttgcagtttgcttctcctgatgacattattttctacagttttaattgaattatgttttaacattttgcttattttttattgtaacctagacaaagtattaaattcatatctatctataggtaccatgtataatattcagcacatgtaggcactgtataatatctaaatcagattaaacatttatctccttaaaacttcagcattttttgatggaatctttcaaaatgctttcttcccatgtttttcttgcactgccctttgttgctagatacagtcaccacaccaggcctgtgtaccctgccactgttttcttcattcccagccaactttaacttagtgcccatccatcaacctcttcctcttcattcccccctctcctagcatctccagccctcatgaggattaatttttccccttggtttattgacaagcctattcattaatatcaatttcagaataattgcttaatcatccactttgctttagttgcatggagaaatttctagtgtaaatattaggaatattaattagataatgaatgaattcatttatgatcatttgggtgacaaggaaaattacacataagatccatgtaaagctgccttccaaattggcctccctagttatgacctagttgtaaaatgagtcactgatccaaaattgaccataatgtgctttattgtcatgcacagttaaacaaaagaaacttgaattagttaatgtattaaaagttgaaactttcatataaaaaatttagattctttttgaatctagaaatggcatcactgggattctggcaacatgagtgggccactcatgtgctgaaatcatgtgttttctcttggttggggtggttggccttcagttccctggttttctttacttactaaaagtcccttccttctccttaaaggccctaaaaacccctgctctagaacttcatcataagcacttactgcaacaagaaaatgtcagtttaaccctccttcgagagctgtatttagttattagaacatagtaaagaaaagcaataaactccaagcactgcaccatcccctgctcatgtgtagtgcaaacaacctaaccaaaccgaaccaaaccaaatcaaactgaatcataacaaaacaaacgtggaaaatccatggtattcttggcatagcccacttagctgttgccaggtgaactagctgtactatgcaaagggagacccaagatcttctggtctaagatctttgcctggatcctggatatacctacactgtgcactatattaatgcatctgtaacttacatagttttcaataatctaatactaaacatttgtagattctaataaactgatcacaatgttgggaagtatgtgatgtaacacaattttccttataagttagtcatgtttctgcctgaagttatcacctgaatcactttgcaggacaaacgctcattacttaaatgatccgaaatgatagcattagttgcctaatgcatacttattattaaataagatatgcatgtatgcacgcatacatacatatttgtatgtatttatttactaaccacatgagcatatgtgctgcttgggctattaacccagcaactaccatgcatgtcttaaattaagcagatattctgtaagatgttggggtatatgaagagcatgagtttttccactgtaaacaatccatggctgctataatgtggttcggaagattcttcttctctttttatttatggaaaatggagccatagtgctgaaacagttttgatttctttctctacacttccttccttgcatcctcccttgttgctctttttcctttccgggaatccaggatatggattgaaacatttaaaatgtctccgttcagtaatagatagtcaccagttatttttatgagttccttgatgctgcacatatgacccagtttagttatcactggggtctgagctggaatacattgctcatcagatacaaagcagtgagagtggcttaggggagccaggtttcccatcagactctcacataaggcttcggtggtgccagatttggctctcagccatgtgagccacacttcaaacagaaatgcacatgaatagttaaagggaaatcctggattcaacctgttgccttctctgtgaagtaatatgtctataatgtctataaattttaataacccagacagcttatttttcaacctaaaactcatgctatcaggaagtttggctgggggtgggtgggcgcagtgcattctctagtgtctctgagactctatctctatgctaatgcaggcccccttgtatgaagttatagtgttctcttatcaaggaacatcaccaccttttacaatctatattttcagtgtgaaagtgtggagatccttgtagaggtctaagaacactggagcattatctgttgattctgccaaagtggcttactctagtgtttacctttgttctccaagatgataactgaaacaaaaattacaaagtgtttcctttggaggttaaagtttctagtggtaataaaagttgaatgaagcaaatcttgagaatcagtataggaacacaaccgtacaagttgagacttaaactgcttatggctataacaccgtgcctcacttgtaagccttgtgtcttagaaacaagggctgtatctcattctctgcacagctggaagacggaagtgttgtaacaagcaaaacacgattttattagttataactatgtttgtgacaagtggccaccataggcattacttgaaattcatctaactatggctcttgaattctggattaatttttgtctattttgggtgggaaggagatagaagttaaaaatcagaatcacatgttctaaagcatttcctacctttttgttttcagtttacaagcactgagaaaggagaaatccagagatgctgctcgatcccgccggggaaaagaaaattttgagttctatgaattggccaagctgttgcctcttcctgccgccatcaccagccagctcgacaaggcatccatcatccgacttacaattagttatctcaagatgagagactttgccaaccaaggggatcctccatggaacctgaggatggaaggccctccaccaaacacctcagtaaaaggtaggttttaggccactgctttgtctggattatggtggtctgtctgtgtctctggaatgaaatgttcaccagcatcctctcttcttttcctgcatatcaaatcctttaaagggattcaaatgtggaaatctgaactctgcatgagaaagacaccatgtgaaggtgacataactctcttctatttctattttgttttagaaaagtctgcccacctaaacattcttttacattaattaagatcttataggagtcaggatgaaataatttacatatgtgttatgatttaggatgaaataatttacatatgtaaatgtgtggggatcatggctcataggtaagagctaatgtgttttgtgttaagtgaacaaaaacagggagagagcaagacccaaccagagcaggtggttttgaattcatgactttattgactttgaaaatcaatccatctattaatttttcatgtttctctatggacacattagctgaaacttatgaatataaacatatatttgtttttcaaatcctttacatagttattcttaaagttatttttaaatagtaggtctgcccctctgatgattaaattttagaattttcttgtttcctgaaacttctaaaaattgagggcatttttagtgttcaaatatgcatggattaattgatgtatatgtggtattggccattgattttcttttcttaagggtgattatttgtagtggatggccaaaaatcttgagattctcatgtcctaacatataaggagcctttgcaagcatcaaataaaaacagactaaggaggtgtagataatctatcctgtaggcaaaaggcagattaagttctttccttaagatttatttttattattttaattgtatgtatgtatatgtgtctctgtggggctatgtacatgtaagtgggggtgccctagaaggcaagaggccttggaccttcctggatctagagtttcaggcagttgtaaatttcccagtgagctggaaaccaaactcaggtcttcttaactgataggacatctctagctctatttttttattgtgggcttcagggtaagacagggtcttattatatagacctggctgttagggatctcactgtgcagaccaggttttcagactcatggagatctgcttatctctgcctaccaagtgctggggttaaaggtttatgccaccatgtccatctgaaataattcttaggtgatgtcatattgccagctaccactgaaaaagtccttgtagctttggctggtgaggtgctataaccttttatttgaattctgatcaaaatgaagcatctattttgaccgtgattgtttgtcattgcatgctaagagctgtgatcacacatatgtgaatcacttagtttgcaaatccctaattacaagccatgaccttagaaactctctagtgctcagagtattccaacatgatgccataataagaaagctttgttgcatgcctattcttatatattaagaataatatataaacttatattaaggtggcatacatagggagatatacatagggatgtgtgaaaaataagtgaattttacatataggcctgactcttttctttcagatattttatgtatatgtaagcattccagcagctgaaatctgatatttctagctgccaatattttggataagaaagacagatgtcatctgtacttaagccgtgtgttcttaggagaaaggggggttgacacaaagtcccacttctaaccaagaaggtgtttgcattggataccagctaggaaagggaaaatgaggtttttttgttttgttttttgttttttgtttttttttctgaaggagtactactgagtatatcagcccaatgtggagaaacaactggccaacacaaaatggactccatgcttttgtgtgccccccccttgttgtttgtttatcttttttttcttttttcttttcttttgttttgaagttagatgggtagggagttcagggaagaggaaaattgattaaaatatatgaaaaatattagataatataaaagaattgtacttggttcgagagaggaaaattagaatttggatttctttatgttattttcctgttgagcagggctgacaaaatttcctataaggaaacttagagtaaatatatttggctttggaattcactcagccctactctcaccaacacacacccaaagtacccctcatgggtaggatgtcaacatgacaaaaactgaacacacattcctgttggctgttctgccttggagaatgtagattatcaacttgctgacataggaatgcatgtcctagataccagcgtaaccctgaaatttcacaaaagatttgtatataattctagtgcatgtgtgttggataaattgtttactctagttcaggcatgattatttttatgtttcatccatacaatgtaaaaggaactgataagtttgttgagtcaggaactacacagcataaaactatattatttaattttatataaattgatagactggacttatatttatttacttagcacaactataatttgcttctgttttgaatgagtttaaaacagaaaaggctctcttggtagggatggcagcccaaatagacaaaagaatgatggatcttgctcattctgttcttgtatgatgaaacagcgtaggtttggaactatggtctttttcagatgtgtacttttagatcatatagaaatattgacttattttacacagataagagatgagtttgcagtggactcatataacagatctaagaccagagttagtgctataaacaacgttcattttgtgtcataattatcaaacattccaggtgagaattttgtcaggcatagactgtaactactaattgatttttcactgtgtggctttcagtcagttcagctctgtaattacctgttatgattaagatgggtaggaagatagagcatggtcacatcccccatgtgtgtcacagtctatgccagggtgcgaggtatcactcatggcagcaggaaggctgggatggttgctgaggagactagaaagagataacacatcttagttaatgattgtggtccagcatggaaattgatacatgttcactgttagtaaactttcgatagacgagagctgaagatgcaagctcagtgactccatcctgtctacactggctctccagtgtctctgggatgcaggatcacttcctacaagctggcactggtgtagaatatgcacacagcttattgctttattctagctctttaatgcaaaactgctactcatttcgatcaaatagtcatgaaacaacaacatcaaaagaaattcattttaatttattaaggtcttctatattgtgcccttgaagaagttcatatggataaacaagcttgtataaatagcctgcagcatcctgtaaagtacagttcataatccaacaataaaacagacagacattatactttgagcctctccagacagcctcttgaataaaccttaaacctaaaggcattttgaaatttatatgaaaagtactcacaccatgtaggcaacattttttaatgtgatgagttgatcatgctaataattaaatttcaaagctgaaatttaaatttgtcccacacaagtatgtagaattttaatcattttaatttgctgcctctgaaaggtcttttactctgcccggcattcatcacatcttttcttagcagtctataaaggttgatgagtttgttgtgtgatgcccattgtatacagattattacaagttatgcttgcagggagaacactcaggtgacagtgtgtaatatgtcccgagtcaattacttcatggaaatgatatgttaagcaattggaaggcaacagatagtttttttctattattttagaaatatctgtattgttatagtacagatcaagtctacaatgaacattatgcattattcacgaaatgaccaggcgttctcggcagtgtttcagctaaataactgatgattgtaacctttagagaaatctcattgtggtgactgttggtaaatcacttctccattcatagtaatctctatttagaggctatctagaagccattacatcttttattaatcttaaaagcaatagctgctgatttgacatttactaaaactagataaattttctatcttcctaatatcatggacttcagtcaatttttggagtatgtctcttgcaactcaattaatctgaggatatttgccattgtacacctaagttttatttcaattcaaacattccagagttttaaatgataatttgcctgcgtgtgtggcagggaatggtgctgaatatataaaatgtatggcagccttagtctctgcagaggagcaattggaaaacaggaagacagctgctgattaaagagcttttctatctttataaaattttactgcaaagagtctcaagagatgcttcatcagtgaggaacattagttgttcttgcagaggacccagttaaattcccagcacccacatggtagcttgcaaccatctctaattccaaactcaggggacccaacattcacttctggcctccatgtgagccatgcatgcatgtgttgaagagtcattattggagatgaagtactcatgcatgtgaggagaaactgacagggctctacttaaacatgctatctggccttattcaaatgccacactcctatgtaaacagtggttcctagcatactcatttaaagtagtcatgtcgaaaggatggactatttaaagactaccccaccaggggatccatcccataatcagccaccaaatgcagacactattgcatatgccagaaagattttgctgaagggaccctgttatagctgtctcgtatgaggctatgccagtgcctggcaaatacagaagtggatgctcacagtcatctattggatggaacacagggcccccaagggagaagctagagaaagcaccaaggagctgaaggggtctgcaactctataagtggaacaacaatatgaactaaccagtacccccagagatcttgtctctagctgcatatgtagcagaagatggcctaatcggccatcactgggaacagaggcctcttggtattacaaactttatatgccccagtacaggggaatgccagggccaagaagcgggagtgggtaggtaggggagcagggtggagggaggttataggaactttcgggatagcatttgaaatgtaaatgaagaaaatatctaataaaaacaaacaaacaaacaaataaataaataacaataaagtaggcatgtaaagcaatgggtacaataatgagtagttttagttttagaagaggtatgggccttctaagatgggctgagcatgtcttctgagtctacacagctgggacacctttgttattactgtcccggcatgttggtgtatgctcataagcatttgcttctagcagctgaccatcatgttaggaggcaagatgttcatccccttccttagaaaccttggaagatgaacgttagtttgtcactctgagctcgggcctgtgtcatcccttgattgtcttttcacataggcaagggtttcaggaccaagagtgaattcttgttacatttggagtagacgcttccttagaaatggacaagcaagtttaaaaatagaaacttggcacttacttggcacttgttcttgcctctgttatcctcaggagattgcaggattctttctgatatctagttccttttgcctcaatccaaagttgcttgtggcaccaaatggaaacatatccagaggcggggctgtcagtgcttattgctgtttgtttgagcaatgtgtgcctggccttagtacccaatggaaatataatattatgtaattgttgttctcaggtatgtttctaggaagcttttttagtgacatactactattcttttctatgtgtatctgaggttacagttttcgtgaagaggtcatttaaaaactaatctttgatttgacttgatttcatgattggacaaggaatgtatgcattgccctccttgcccaggttcattctctcgtagtggggagagcaagatatttgacaagtgggggcaaaatgatggggaaaaatatatttctaagattcttttggagtttcacactattgtgaaaagtttgtctaaatatgtggactgtgatatatggaatcagaacataatttagcatgaaaatattgtgtgatatgtttaagttttgtgtgtgtgtatgtacatgtgcgagtgtgtgtgagcatgagagtgtgtgtatgtgttttgctatatgtcactgtttgactcattggccccatttcttgtgtatatgttgtgactggacatctagaattatcttcaatcccataccctgaaggaaagcttatttggaagaattatcactgagaatgtgtgactctcaaggtgtgcttgtcagttacttacagggaagaagaactgatcctatcttcatagtgcagtggttcataaatggtatagggccacttatactatcatgaattgagtcagagtaaggcaagtccatggccagcaactctcagtggacctcttttggaagctaccatgaagaatcatggaaaagacatggatacatgataaaagcacaggacttactagaatattctctccatggtattgtttctagtactttattcttgtcttctggatttgtttttgccaaaaaggaaaattggatacttggaggggagggaggcatttctggaaatgtgagcacaaaaatgaacatgagctcaatctgccctctctccactatcatctcatttttcaatagcaatcagtgctgcaagccatctatgtatgttttatatatgtcactattatactaaaatgtcctccttgtagaaacacacacacattcatactcacagactcccctttccctccctcatctaggacaagtaccatggctggcattcccataacaaagtcaccaagagaatattggaacacattcactttaaccaaattttatgtgatcctgaaagccttcaataaaaaagacataaacacatgcagaagactatttattctcatttccagtctcagtaaagactggacagcacatatggatatgactagatgatatgacatgccttatgtttttagattcctctagattcttctcagcctgtctgttcaacatggggcacagggaaagccacctgccactgagagactttggagaacaaagggcaatgaaggccagagagaacatctgttttctaaagcttttgtactcttgttcatgttaaagttttgtgctttgaaatttgtttcctgagttcactatagctggtgagtttggtgtgttcctgttcagcttttgatacccagtccatatgtcccattcttatgtaaaattctcttcctcctgaatcattagcaatctttgtgaggcacactgacattttttctgtcactttattgttagcctatcctttatgtacttttatgtacttgcctgtggactagggcataagatcattgaaagcattgtctggctccttttgttcattttaacagctaagaatgctgctttgtacctggaagagtactgagtcctgctttagaattgaattatagtaaagatttctgttttcacacttaaataatactcatcgaatgctatgtttgagcagctgtgggcaggtgaaaataataaaagtggaagaataaaaagaaaaaataggaaaactggagggagaacaatggttatagtgtgaaatcaaaggaaaagtaatttgggttgtagagcaggcatgagagggagtggaagttcttgcatgagcccttgtgatcccattggcttactcacagatgcatggggaggagtggaaaatcccagtctcacaatgcccacaagaaccagtgtaacctgaagtctggtcagcagatttttgtcttttctcatatttaatttagtgctccacattagagtgttcacattttctagaaggagaaggagagggagagagagagagagagaaagagagagagagagagagagagagagagagagagagagagagagagagagagagagggagagagggagagagggagagagggagggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncatccatccatccatctactttctaattttgaattcaaattttgtagaaaagatttatcaattagtatcttctgagatggctcaggtttcttgaagctgtgtattattagtatcacaacctatgtactttcactttgtatttgctaatatctctgtcttttattttactttattcccctcaacataaggatattttatatctctatcatgtcttggactaaaaatatgaccagcacacatgagcacatggaaacatgtacatgaatacgcatacacattttcccactgaagtcttttaaatctgttttgatcattgcgatttgaaaagatgtaatcattacaaatcagagtctaaaaacaaagcacagagttttgtaaaaatgtagcatccacagactaggtccctgggactgtatatgtttgttattaacttttactttcttggcataactttaaggcacactcttgtattctttacatttgactttgaaaatctactggttcctaaataatgtcatgcatcggtcatggcaccctgtggcactatcttatgctgtttggtatccacttatttagtactttcaagtttctgtgctctgttttgtaagactggaagtcttgaatatgcttttgttggccctataacctcttgcaaagcataatatattgtttcagcactctgtggataacaatatttcatgagcatagcaaagagtcacctctgactgctttatatgattgaaattgtactatgactttaatttcccgaaactgcttcctagatactgtgcataagtcttagctgtgccttaataaacatgtggtgacctctacctatgaaactaatgtggctttgaatactgattggctacagcttttcacatcatttttgagtctgcagaatgtcaataaatgccagaacctaggccaaaaaggagcgtaaagagaattttttgatctatattttttagaagccaggcttttatcccattcttgttttgaaaaagaaagaaagaaagaaggaaaaaaagaaagaaagaaagaaagaaagaaagaaagaaagaaagaaagaaagaaagaaagaaagaaagaaagaatttgagagaaggtagaaagaagttcaatgacaagataaagtgattactgatcagtggcctgtagctctttgtgt